(19) United States
(12) Patent Application Publication
(10) Pub. No.: US 2010/0191037 A1
(43) Pub. Date: Jul. 29, 2010

(54) ISO MUSIC THERAPY PROGRAM AND METHODS OF USING THE SAME

(76) Inventors: Lorenzo Cohen, Houston, TX (US); Michael M. Richardson, Houston, TX (US)

Correspondence Address: Nielsen IP Law LLC, 1177 West Loop South, Suite 1600, Houston, TX, 77027, US

(21) Appl. No.: 12/602,448

(22) PCT Filed: Jun. 2, 2008
(86) PCT No.: PCT/US08/65505

§ 371 Date: Apr. 2, 2010

Related U.S. Application Data
(60) Provisional application No. 60/941,540, filed on Jun. 1, 2007.

(51) Int. Cl. A61M 21/00; G09B 19/00; G06F 3/048

(52) U.S. Cl. 600/28; 434/236; 707/722; 715/716

(57) ABSTRACT

The use of music in connection with cancer therapy and other conditions is described. A computer-implemented method of modulating a mood in a person includes selecting an appropriate target mood, filtering a play list of music tracks based on an initial mood and vectoring by further filtering the play list to allocate the music tracks to play in order of appropriate stages creating a progressive modulation of mood from the initial mood to the target mood. A method of treating a patient with debilitating condition by administering to the patient a computer-implemented methodology is also provided.

### CROSS-REFERENCE TO RELATED APPLICATIONS

This application claims priority to U.S. Pat. App. Ser. No. 60/941,540 filed Jun. 1, 2007. The application is incorporated by reference herein in its entirety.

### FIELD OF INVENTION

The use of music in connection with cancer therapy and other conditions is described. Special software programs organize music in a therapeutically useful fashion or to meet the user's psychological and physical needs.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

Stress has been associated with an increased likelihood of upper respiratory tract infections, reactivation of latent viral infections, and increased wound-healing time. Kiecolt-Glaser, J. K., Chronic Stress and Immunity in Family Caregivers of Alzheimer's Disease Victims, Psychosomatic Medicine 1987, 49:523-35; Glaser, R., et al., Stress, Loneliness, and Changes in Herpesvirus Latency, Journal of Behavioral Medicine 1985, 8:249-60; Cohen, S., et al., Psychological Stress and Susceptibility to the Common Cold, New England Journal of Medicine 1991, 325:606-12; Cohen, S., et al., Negative Life Events, Perceived Stress, Negative Affect, and Susceptibility to the Common Cold, Journal of Personality & Social Psychology, 1993, 64:131-40; Stone, A. A., et al., Development of Common Cold Symptoms Following Experimental Rhinovirus Infection Is Related to Prior Stressful Life Events, Behavioral Medicine 1992, 18:115-20; Kiecolt-Glaser, J. K., et al., Slowing of Wound Healing by Psychological Stress, Lancet 1995, 346:1194-6; Marucha, P. T., Mucosal Wound Healing Is Impaired by Examination Stress, Psychosomatic Medicine 1998, 60:362-5. Stress-induced physiological changes include persistent increases in SNS activity that can cause increased blood pressure, heart rate, catecholamine secretion, and increased platelet aggregation.

Stress can have profound physiological effects in cancer patients as well. The diagnosis and treatment of cancer, in particular, are associated with distress and the fear of disease progression, recurrence, and death. Cancer patients are also apprehensive about decreases in their quality of life (QOL), including side effects of treatment, physical changes, and changes in social relationships. These cancer-related stressors, together with other sources of stress, can have negative physical and emotional health consequences for patients with cancer. Depression is a predictor of mortality in an otherwise healthy population, and in a recent randomized trial, patients with depression who received conventional treatment for depression lived significantly longer than those who did not receive treatment for their depression. Gallo, J. J., et al, The Effect of a Primary Care Practice-Based Depression Intervention on Mortality in Older Adults A Randomized Trial, Annals of Internal Medicine 2007; 146:689-698.

Stress-induced physiological changes can affect cancer progression, treatment, recovery, and recurrence. Several studies have linked stress and other psychosocial factors to the incidence and progression of cancer. Gehde, E., et al., Early Experience and Development of Cancer in Later Life: Implications for Psychoimmunologic Research, International Journal of Neuroscience 1990, 51:257-60; Morris, T., et al., Patterns of Expression of Anger and Their Psychological Correlates in Women with Breast Cancer, Journal of Psychosomatic Research 1981, 25:111-7; Greer, S., et al., Psychological Response to Breast Cancer: Effect on Outcome, Lancet 1979, 2:1239-48.

In addition, recent research has demonstrated that depression, which is a common psychological response to stressful life events or circumstances, has been linked to an increased risk of cancer, progression of disease, and lower rates of survival. Penninx, B. W., et al., Chronically Depressed Mood and Cancer Risk in Older Persons, Journal of the National Cancer Institute 1998, 90:1888-93; Watson, M., et al., Influence of Psychological Response on Survival in Breast Cancer: A Population-Based Cohort Study, The Lancet 1999, 354:1331-6; Strommel, M., et al., Depression and Functional Status as Predictors of Death Among Cancer Patients, Cancer 2002, 94:2719-27; Loberiza, F. R., et al., Association of Depressive Syndrome and Early Deaths Among Patients After Stem-Cell Transplantation for Malignant Diseases, Journal of Clinical Oncology 2002, 20:2118-26. It has been established that stress and depression cause suppression of cell-mediated immunity. Irwin, M., et al., Reduction of Immune Function in Life Stress and Depression, Biological Psychiatry 1990; 27:22-30; Rabin, B. S., Stress, Immune Function, and Health: The Connection, New York, N.Y.: Wiley-Liss & Sons, 1999.

A stress-induced decrease in natural killer (NK) cell activity, a primary defense against metastatic tumor growth, has been associated with tumor growth in animals. Stefanski, V., et al., Social Confrontation and Tumor Metastasis in Rats: Defeat and B-Adrenergic Mechanisms, Physiology and Behavior; Brenner, G. J., Sympathetic Nervous System Modulation of Tumor Metastases and Host Defense Mechanisms, Journal of Neuroimmunology 1992, 37:191-202; Ben-Eliyahu, S., et al., Stress Increases Metastatic Spread of a Mammary Tumor in Rats: Evidence for Mediation by the Immune System, Brain, Behavior, and Immunity 1991, 5:193-205. Extensive research has demonstrated that depression is a predictor of mortality in an otherwise healthy population, and in a recent randomized trial, patients with depression who received conventional treatment for depression lived significantly longer than those who did not receive treatment for their depression. Gallo, J. J., et al, The Effect of a Primary Care Practice-Based Depression Intervention on Mortality in Older Adults A Randomized Trial, Annals of Internal Medicine 2007; 146:689-698.

In light of the foregoing, methods for effecting psychological changes in patients under duress, such as those with debilitating mental illnesses or life threatening diseases such as cancer, or people simply experiencing the stress of daily life, through modulation of mood would be beneficial.

### SUMMARY OF THE INVENTION

A computer-implemented method of modulating the mood in a person disclosed herein includes selecting an appropriate target mood, filtering a play list of music tracks based on an initial mood and vectoring by further filtering of the play list to allocate the music tracks to play in order of appropriate stages creating a progressive modulation of mood from the initial mood to the target mood.

Disclosed herein is a computer software program that interfaces with other preexisting software programs such as Itunes or windows-based music library programs. The program can arrange music in a library in a predetermined order based on the beats per minute of the music (BPM—tempo). The user can organize the music manually or the program will organize the music automatically based on a pre-specified outcome that is desired.

The first step of a program disclosed herein is to tag all tracks in the user's library according to the tracks BPM. This is accomplished through the use of preexisting programs such as: Tangerine BPM, beaTunes, MixMeister BPM Analyzer, and BPM Detector Pro. These programs compute BPM automatically and save the information as files or ID tags.

Once all the music is tagged, the user needs to decide if they want to manually organize their tracks based on BPM for the desired outcome or to use the program. The program can either be fully automatic or the user can choose specific tracks within pre-filtered portions of the library for a specific desired effect. Once the music is appropriately tagged with BPM, the user can also organized the music and create a play list separate from the algorithms of the program.

The user can organize their music library and create play lists for specific desired effects. This could include to help them relax, to lift their spirits, to help them get going, to motivate them while exercising, and others.

The program follows the basic rules of the ISO principle where the user will choose how they want to use the music (e.g., to help them relax, to lift their spirits, to help them get going, to motivate them while exercising, and others). They will first choose music based on BPM to match their mood. For example, if a patient is stressed, the first tracks will have high BPM. Music of progressively slower tempos is then used to move the patient to a more relaxed state.

Users will enter the stage of music that matches their mood based on their report of mood. For example, they could indicate how stressed or relaxed they are on a sliding scale. With the programmed logic, the software program will be able to allocate music tracks to appropriate stages to allow the user to either become relaxed or less depressed/lift their spirits. The program can also select music that matches a high tempo and stays at that stage or varies according to pre-specified logic (e.g., for exercise) or matches a low tempo and stays at that stage (for continued relaxation once relaxed). The user can also perform these choices on their own.

In the manual mode, the user chooses tracks to create a play list for their desired effect. In the semi-automatic mode (user interfacing with computer), the user will work with the program to create a play list for the desired effect. For example, if the user indicates they want to use the program to help them relax they will first indicate if they want to filter the music library based on one or more genres of music. They will next indicate their mood state. The program will then filter the library of music based on BPM and allow the user to select a number of tracks that have BPM that match what they indicated their stress level was. For example, if the user indicated 10 out of 10 on stressed they would choose from fast tempo music to begin with. If they indicated 5 out of 10 on stressed they would choose from moderate tempo music to begin with. These are just some examples. The songs they choose will be organized based on BPM with the slower tracks coming after the faster tracks.

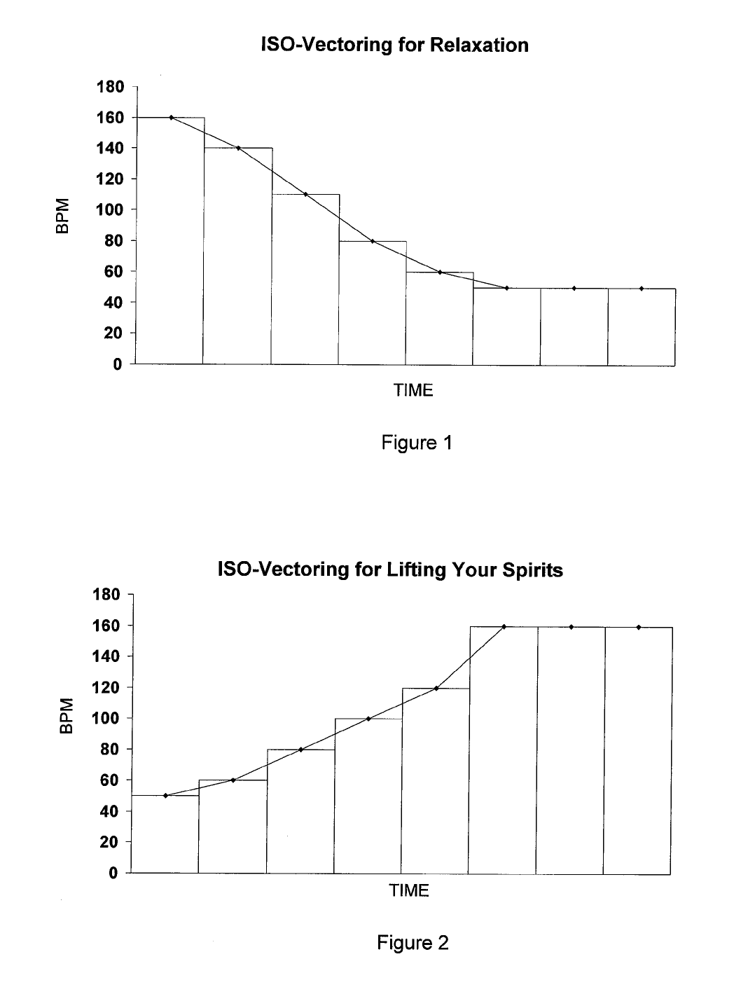

Once a few tracks are chosen, they will move to the next stage where the music library will be filtered again based on tracks that match a slower BPM than the first few tracks chosen. The user will continue choosing tracks that are progressively slower and slower until they have created a music play list of a desired length (see FIG. 1). The tracks available at each stage are filtered for the user and the user needs to simply choose the track they want. The tracks will always be organized from faster to slower BPM. This is just one example.

The user can also create a play list to lift their spirits when feeling down and blue. The interaction with the program would be the same as when helping them to relax, except in this case the user would first choose songs that are of a slow BPM and then progressively faster BPM until they have their play list of the desired length (see FIG. 2). The tracks at each stage are filtered for the user so they only can choose tracks appropriate for each stage of the vectoring.

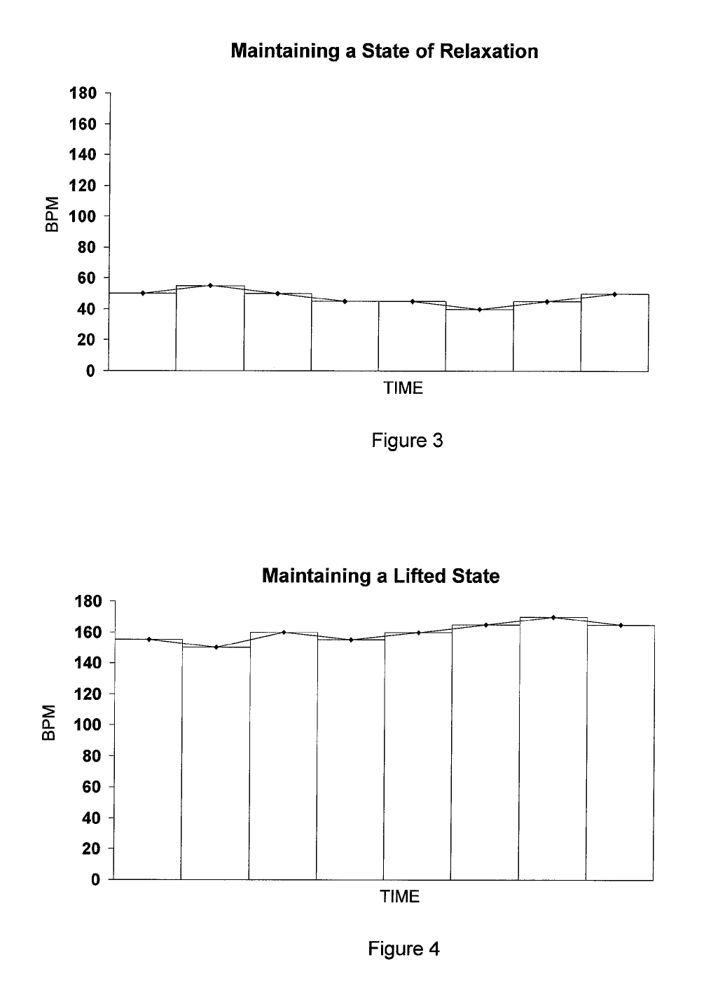

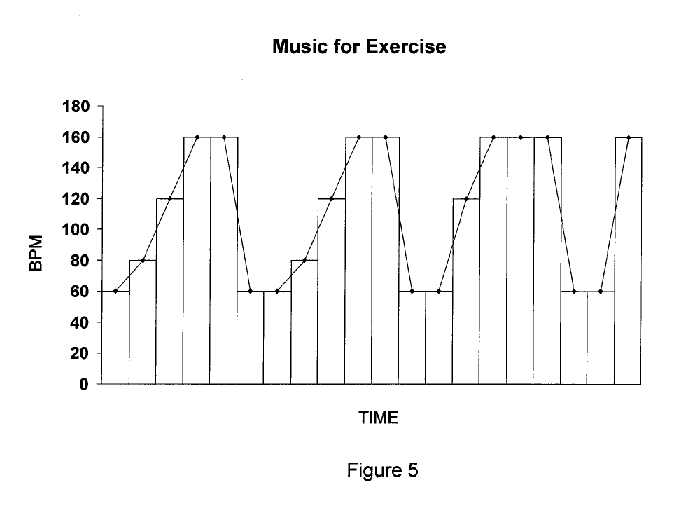

There are other examples of how the user can use the program such as choosing music that is of slow BPM to continue relaxation (see FIG. 3) or some other mood state (see FIG. 4), choose music that is fast BPM for exercise or other activity. In addition, the vectoring can take on different shapes, not just fast to slow BPM or slow to fast BPM. It could start slow and move to fast and then become moderate and then fast again, etc. This could be useful for different forms of exercise (see FIG. 5).

The user can also have the option of interfacing with the program and having the music selection be fully automatic. Once the user indicates what they want to make the play list for (to help them relax, to lift their spirits, to help them get going, to motivate them while exercising, and others), they will indicate their mood if appropriate for that selection, and the program will automatically select tracks based on BPM to appropriately vector the user. If the user wants to modify any of the tracks they can simply remove and add tracks. This will put them into the semi-automatic mode of the program.

The play list length for helping people to relax, lift their spirits, or change their mood in some other way is 25-35 minutes. The user can choose, however, to make a play list of their desired length.

The play list for helping people to relax, lift their spirits, or change their mood in some other way may have fewer tracks at the beginning of the play list (stages 1 and 2) and more tracks near the end of the play list (stages 4 and 5). This will allow the user to have a greater amount of music matching the desired mood state at the end of the play list (e.g., slow BPM for relaxation, fast BPM to lift the spirits, etc.).

In other aspects, methods disclosed herein provide for the use of the computer-implemented method as part of a treatment regimen for patients experiencing debilitating conditions such as stress, depression, and life-altering diseases associated with high anxiety and fear such as cancer.

In other embodiments disclosed herein is a computer-readable medium containing instructions for controlling a computer system that can carry out the method of organizing music for modulating the mood of a person.

The foregoing has outlined rather broadly the features of the embodiments disclosed herein in order that the detailed description of the embodiments disclosed herein that follows may be better understood. Additional features and advantages of the embodiments disclosed herein will be described hereinafter, which form the subject of the claims of the embodiments disclosed herein.

### DETAILED DESCRIPTION OF THE DRAWINGS

FIG. 1 is a graph of beats per minute over time where user will continue choosing tracks that are progressively slower and slower until they have created a music play list of a desired length.

FIG. 2 is a graph of beats per minute over time where the user would first choose songs that are of a slow BPM and then progressively faster BPM until they have their play list of the desired length.

FIG. 3 is a graph of beats per minute over time where the user uses the program for continued relaxation.

FIG. 4 is a graph of beats per minute over time where the user uses the program of an embodiment disclosed herein for maintaining a lifted state.

FIG. 5 is a graph of beats per minute over time where the user uses the program for exercise.

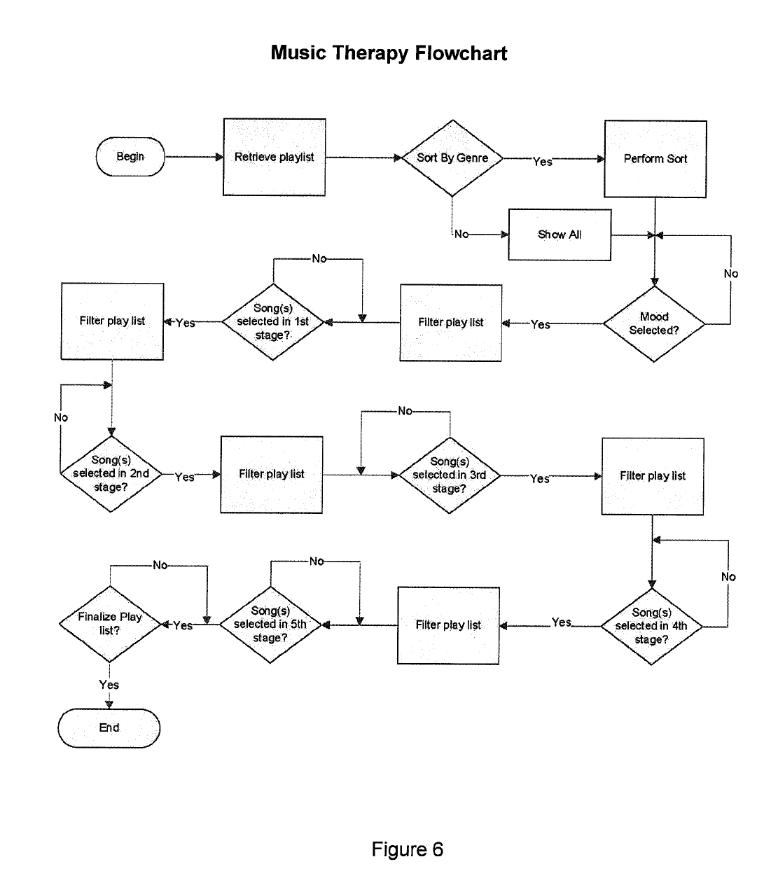

FIG. 6 shows a flow chart of the methodology of a single embodiment used in connection with an embodiment disclosed herein.

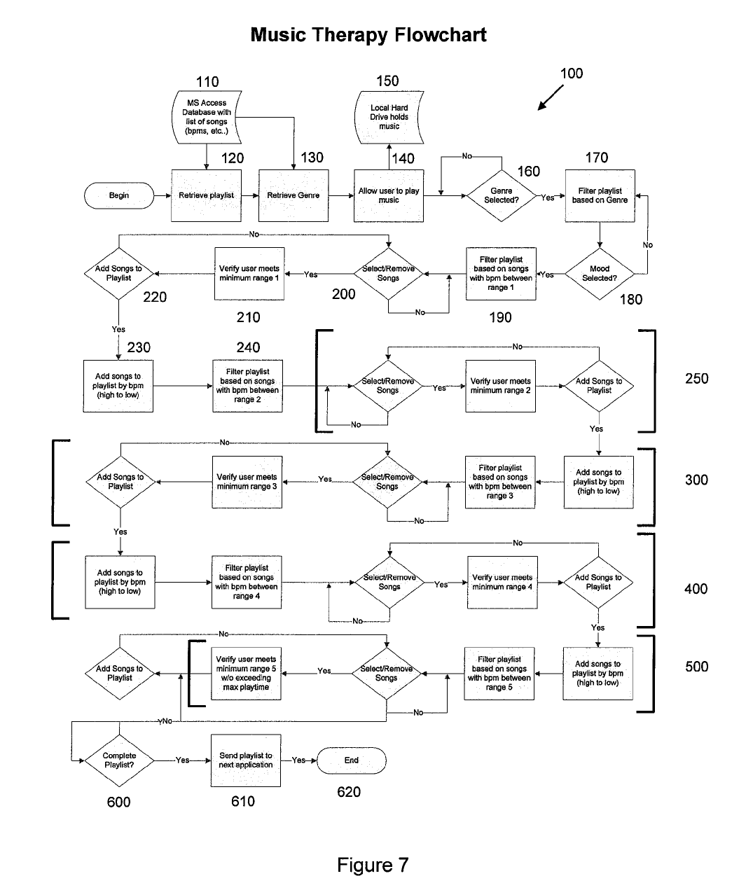

FIG. 7 shows a flow chart of the computer-implemented method for modulating the mood of a person.

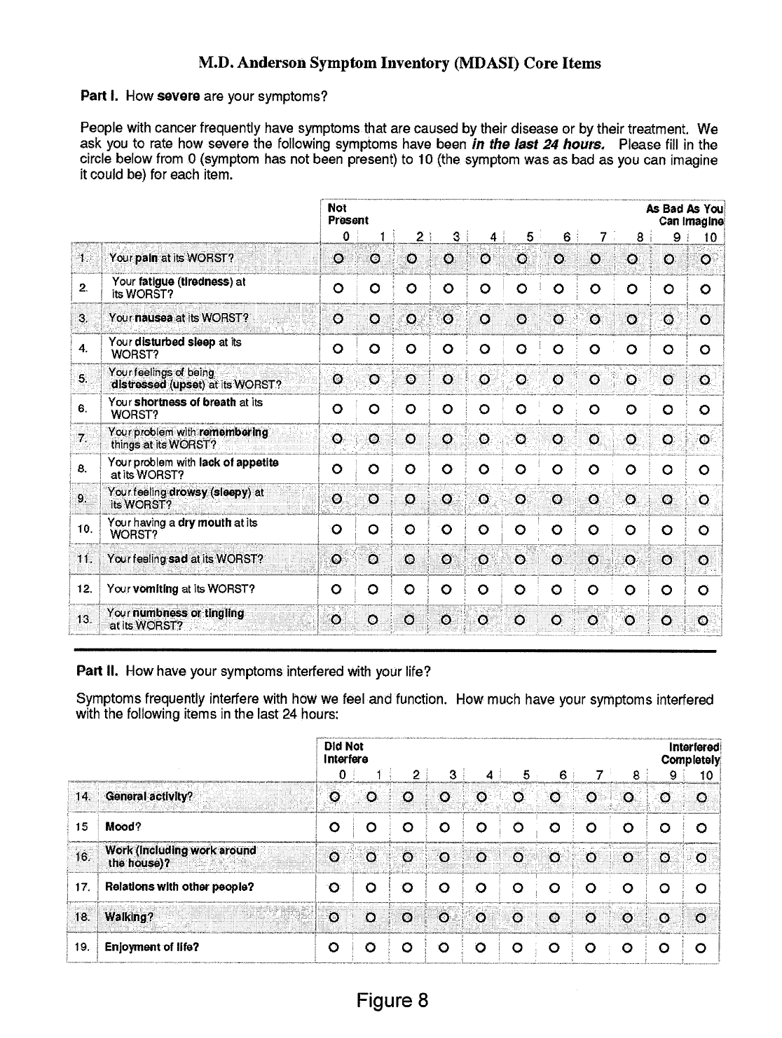

FIG. 8 represents a questionnaire useful in the MDASI analysis.

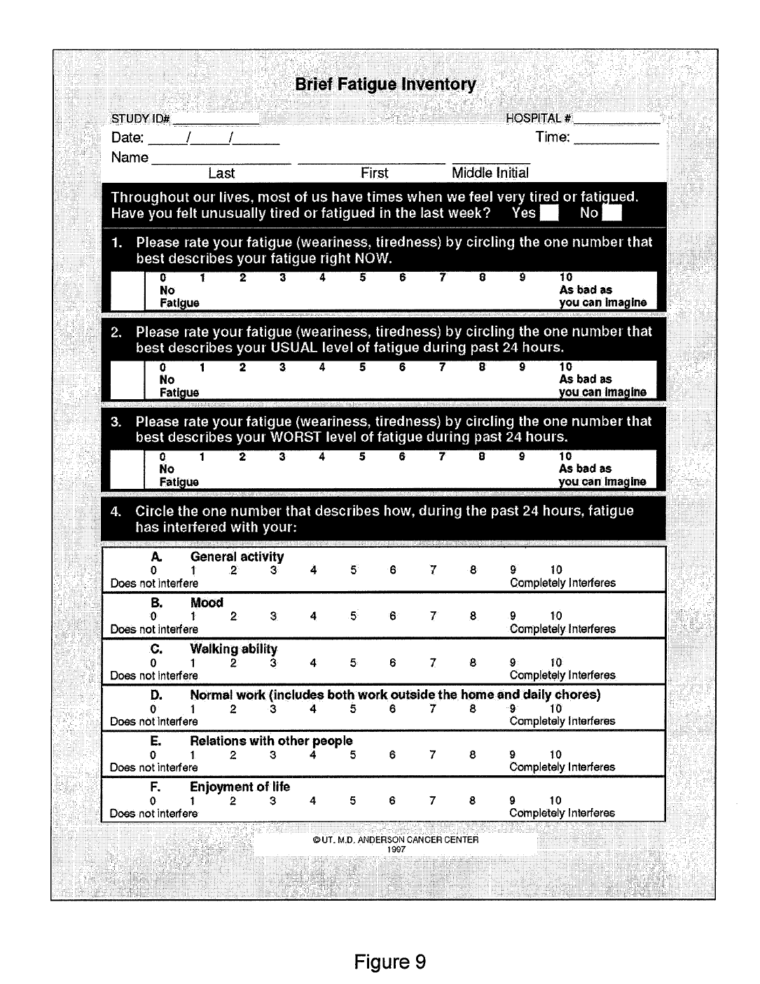

FIG. 9 represents a questionnaire useful in the Brief Fatigue Inventory analysis.

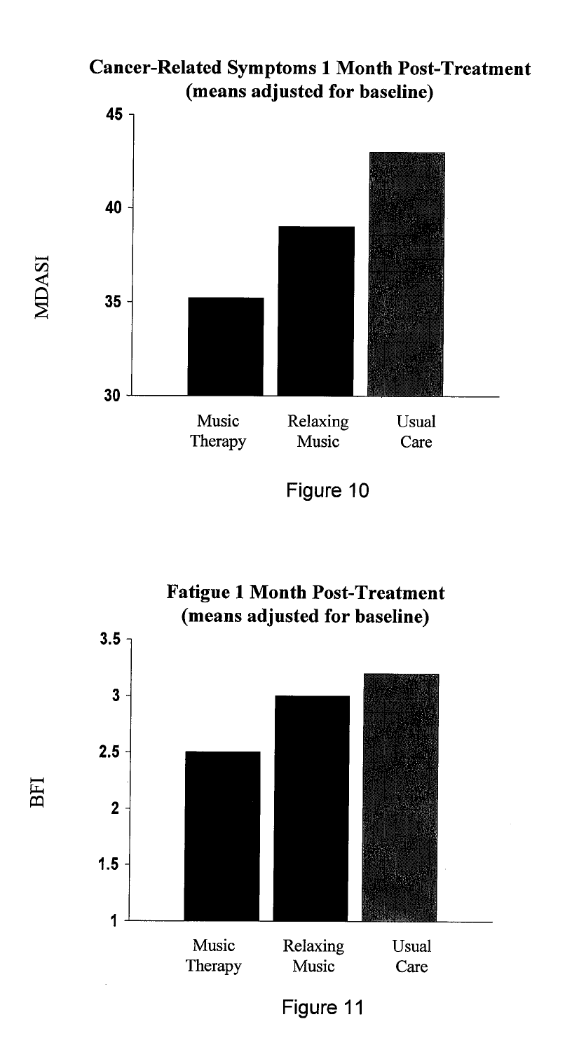

FIG. 10 depicts MDASI data one month after cancer treatment.

FIG. 11 depicts BFI data one month after cancer treatment.

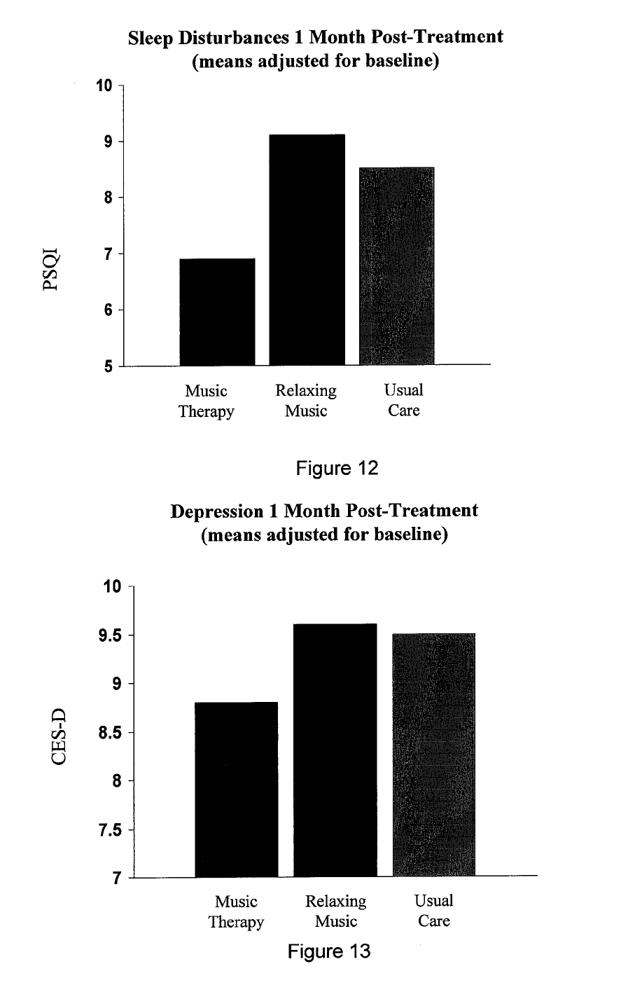

FIG. 12 depicts PSQI data one month after cancer treatment.

FIG. 13 depicts CES-D data one month after cancer treatment.

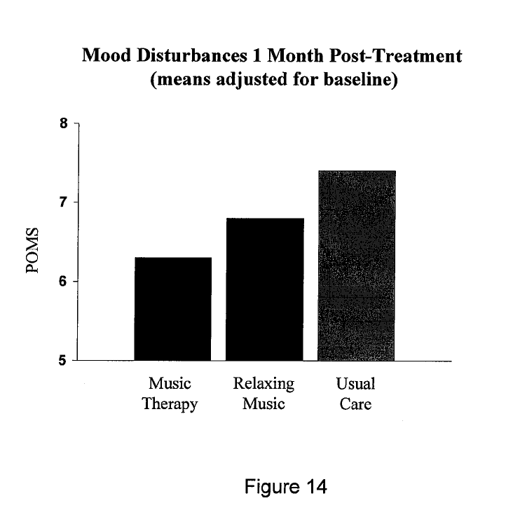

FIG. 14 depicts POMS data one month after cancer treatment.

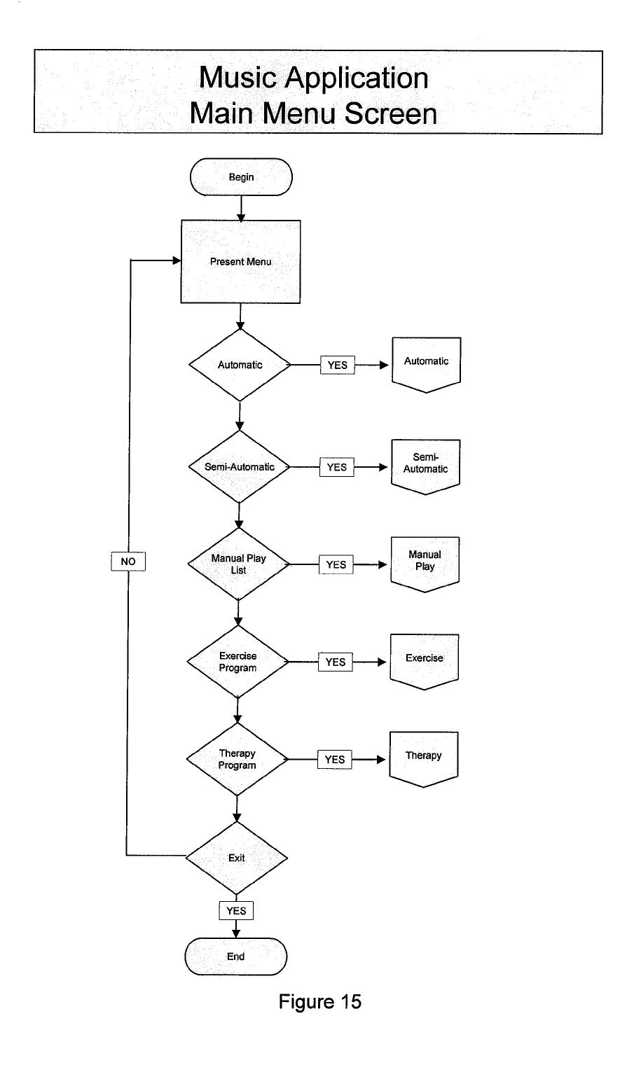

FIG. 15 is a flow chart of a main screen useful in connection with the methodology disclosed herein.

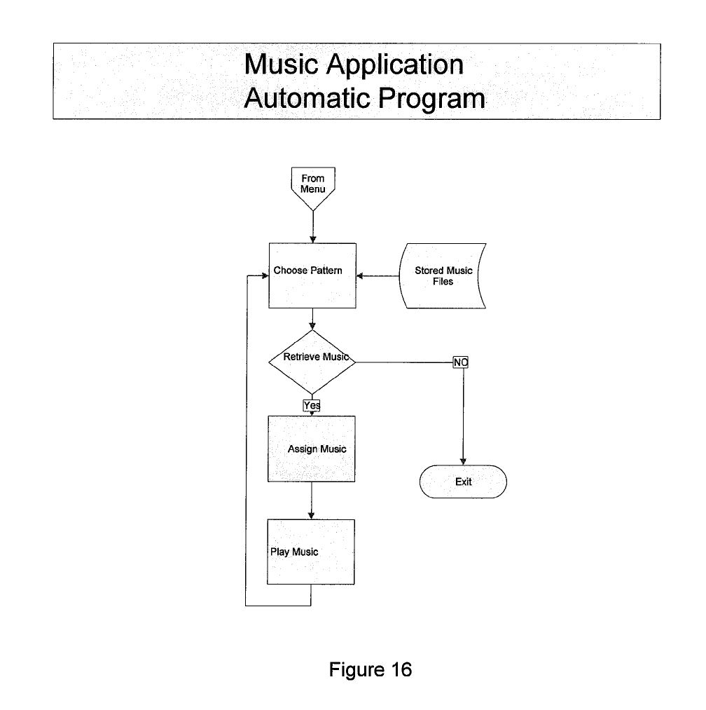

FIG. 16 is a flow chart of the methodology of an automatic program.

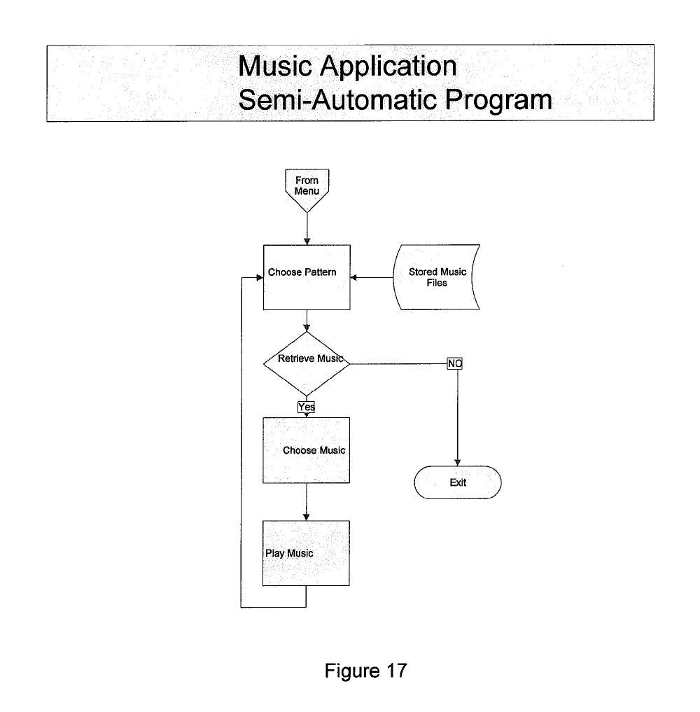

FIG. 17 is a flow chart of the methodology of a semi-automatic program.

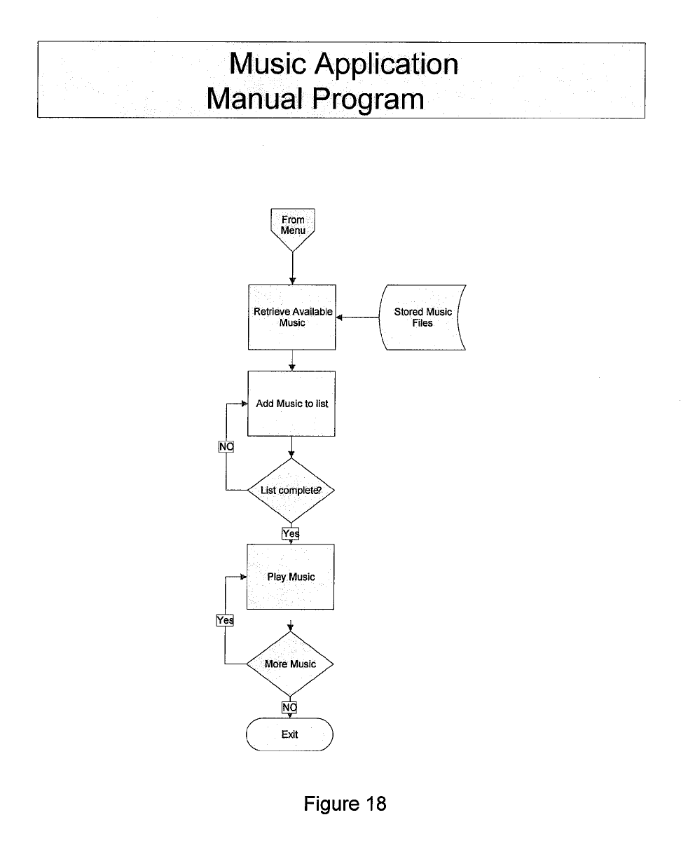

FIG. 18 is a flow chart of the methodology of a manual program.

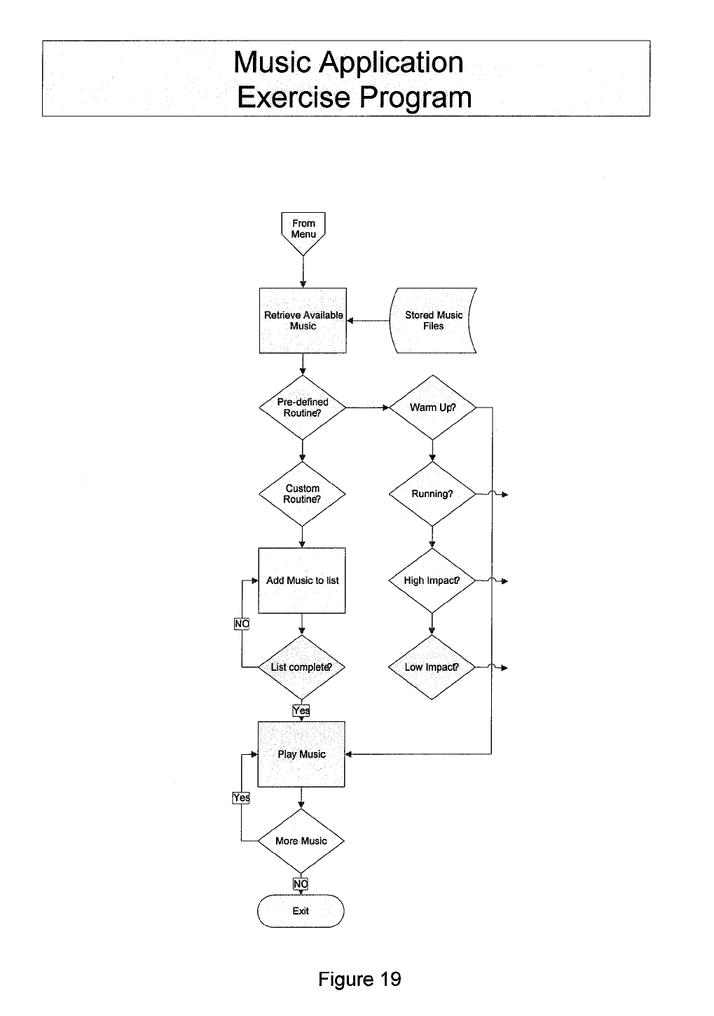

FIG. 19 is a flow chart of the methodology of a music application for exercise program.

### DETAILED DESCRIPTION

A method disclosed herein is a computer-implemented method of modulating a mood of a person comprising the steps of selecting an appropriate target mood, filtering a play list of music tracks, and vectoring by further filtering the play list to allocate the music tracks to play in order of appropriate stages creating a progressive modulation of mood from an initial mood to the target mood, wherein said music tracks are sorted by music tempo in beats per minute.

Another method disclosed herein is a computer-implemented method of modulating a selected state in a person comprising the steps of selecting an appropriate target state, filtering a play list of music tracks, and vectoring by further filtering the play list to allocate the music tracks to play in order of appropriate stages creating a progressive modulation of the selected state from an initial state to the target state, wherein said music tracks are sorted by music tempo in beats per minute.

Another method disclosed herein is a computer-implemented method of organizing music tracks based on music tempo in beats per minute comprising the steps of selecting a pattern, filtering a play list of music tracks, and vectoring by further filtering the play list to allocate the music tracks to play in order of appropriate stages creating a change in music tracks according to user preference, wherein said music tracks are sorted by music tempo in beats per minute.

Another method disclosed herein is a method of administering a computer-implemented methodology to modulate moods, symptoms, fatigue, and sleep quality of a patient undergoing cancer treatment comprising the steps of selecting an appropriate target mood, filtering a play list of music tracks, and vectoring by further filtering the play list to allocate the music tracks to play in order of appropriate stages creating a progressive modulation from an initial mood to the target mood, wherein said music tracks are sorted by music tempo in beats per minute.

Another method disclosed herein is a method of modulating stress in a patient with a debilitating condition and having an initial mood comprising the steps of (a) determining the target mood for the patient, (b) filtering a play list of music tracks stored on a computer, (c) vectoring the play list by further filtering the play list to allocate the music tracks for a progressive modulation of mood from an initial mood to the target mood, (d) monitoring psychological responses to determine stress level, and (e) repeating steps a through d until the target mood is reached.

Disclosed herein is a computer-readable medium containing instructions for controlling a computer system to carry out a method to modulate a person's mood, the method comprising selecting a target mood, filtering a play list of music tracks, vectoring by further filtering the play list to allocate the music tracks to play in order of appropriate stages creating a progressive modulation of mood from an initial mood to the target mood, wherein said music tracks are sorted by music tempo in beats per minute.

Disclosed herein is another computer-readable medium containing instructions for controlling a computer system to carry out a method to organize music according to user preference for some desired target state, the method comprising selecting a target mood, filtering a play list of music tracks, vectoring by further filtering the play list to allocate the music tracks to play in order of appropriate stages creating a progressive modulation of state from an initial state to the desired target state, wherein said music tracks are sorted by music tempo in beats per minute.

Disclosed herein is another computer-readable medium containing instructions for controlling a computer system to carry out a method to organize music tracks based on music tempo in beats per minute, the method comprising selecting a target mood, filtering a play list of music tracks, vectoring by further filtering the play list to allocate the music tracks to play in order of appropriate stages creating a change in music tracks according to user preference, wherein said music tracks are sorted by music tempo in beats per minute.

Disclosed herein is another computer-readable medium containing instructions for controlling a computer system to carry out a method to modulate a mood of a patient with a debilitating condition, the method comprising selecting a target mood, filtering a play list of music tracks, vectoring by further filtering the play list to allocate the music tracks to play in order of appropriate stages creating a progressive modulation from an initial mood to the target mood, wherein said music tracks are sorted by music tempo in beats per minute.

Disclosed herein is a program storage device readable by a computer, embodying a program of instructions executable by the computer to perform the steps for modulating a mood of a person wherein said program comprises the steps of selecting an appropriate target mood, filtering a play list of music tracks, vectoring by further filtering the play list to allocate the music tracks to play in order of appropriate stages creating a progressive modulation of mood from an initial mood to the target mood, wherein said music tracks are sorted by music tempo in beats per minute.

Disclosed herein is another program storage device readable by a computer, embodying a program of instructions executable by the computer to organize music according to user preference for some desired target state, wherein said program comprises the steps of selecting a target mood, filtering a play list of music tracks, vectoring by further filtering the play list to allocate the music tracks to play in order of appropriate stages creating a progressive modulation of a state from an initial state to the desired target state, wherein said music tracks are sorted by music tempo in beats per minute.

Disclosed herein is another program storage device readable by a computer, embodying a program of instructions executable by the computer to organize music tracks based on music tempo in beats per minute, wherein said program comprises the steps of selecting a target mood, filtering a play list of music tracks, vectoring by further filtering the play list to allocate the music tracks to play in order of appropriate stages creating a change in music tracks according to user preference, wherein said music tracks are sorted by music tempo in beats per minute.

Another method disclosed herein is a method of treating a patient with debilitating condition by administering to the patient a computer-implemented methodology comprising the steps of (a) determining an initial mood of the patient, (b) determining the target mood for the patient, (c) filtering a play list of music tracks stored on a computer, (d) vectoring the play list by further filtering the play list to allocate the music tracks for a progressive modulation of mood from an initial mood to the target mood, (e) monitoring psychological responses to determine stress level, and (f) repeating steps b through e until the target mood is reached.

The computer-implemented method of modulating mood may be stored on computer-readable medium containing instructions for controlling a computer system to carry out the method. The program storage device readable by a computer, embodying the computer-implemented method may be a disk or a hard drive, for example.

Advantageously, patients with cancer and other debilitating conditions that may be associated with stress, depression, and general duress, that use the computer-implemented methods described herein may experience changes in their mood or quality of life. Adjusting to many of the psychological and physical changes associated with cancer, in particular, can be overwhelming, but stress management interventions via psychosocial and psychoeducational support improve mental health and quality of life, in addition to improving immune status, pain indices, and, in some cases, length of survival and decrease hospital costs.

The use of music to help change people's moods is pervasive throughout the world and has been used for millennia. Music is used to help people relax, to cheer people up, to make them happy, to illicit deeply felt emotions, and more recently, to help motivate people when they are exercising. Music can affect psychological and physiological changes in individuals.

The form music therapy takes varies depending on the individual and their circumstances. A music therapist is a qualified practitioner who has undergone rigorous training specializing in using music as a therapeutic tool. They are trained to assess each patient with regard to their problem and their experience with music. The therapist then decides what mode of therapy would benefit the patient based on music therapy principles and techniques. Since the therapy is individual to the patient's situation and experience with music, the modality of therapy can vary. Therapies can range from listening to music, actively creating music with instruments, talking about music, and lyric writing among others.

One such music therapy technique uses the “ISO” principle. The ISO (which is derived from the Greek word for “equal to”) principle has been used in music therapy to stimulate a change in a negative affect. Altshuler I. M., A Psychiatrist's Experience with Music as a Therapeutic Agent, Music and Medicine, eds., Schuillian et al., Book for Libraries Press (1948). Music is selected that is “equal to” or matches the initial mood or mental tempo of the subject. The music tracks played at the start of the therapy are designed to match the initial mood of the person. The initial mood is used as a guide to determine the musical tempo of the music tracks at the start of the therapy. For example, if a patient is anxious, fast tempo music would be used to begin the transitioning. Music of progressively slower tempos and calmer content would be used to move the patient to a more relaxed state. This would result in the subject becoming more relaxed. This is what is known as “vectoring.” This form of vectoring is thought to be more effective than simply listening to calm peaceful music when the user is still feeling stressed. By gaining the attention of the subject enable a music therapist can transition the patient to another mood. The same technique can be used with other moods such as depression. It could also be used to help motivate people while exercising or just helping them to get going. These are just some examples of how the ISO principle can be used.

In order to establish an appropriate music therapy regimen a user must select a desired target mood. Therefore, the computer-implemented method may be operable to interface with a user who is a clinician, a doctor, a person seeking the therapeutic effect of the music, and any combination thereof. The target mood is generally going to be a positive mental state such as states of relaxation, happiness, contentedness, well-being, and uplifted spirit.

The user can organize their music library and create play lists for specific desired effects. This could include to help them relax, to lift their spirits, to help them get going, to motivate them while exercising, for example. The program follows the basic rules of the ISO principle where the user will choose how they want to use the music (e.g., to help them relax, to lift their spirits, to help them get going, to motivate them while exercising, etc.).

While working for the Texas Department of Corrections (currently Texas Department of Criminal Justice) as a music therapist, Michael M. Richardson, MT-BC used live and recorded music following the ISO-Vectoring Principle with psychiatric patients to improve their mood. In 1993, he began working on the inpatient pediatric unit at The University of Texas M. D. Anderson Cancer Center and observed children undergoing bone-marrow aspirations and lumbar punctures. In an effort to ameliorate anxiety and create a calming environment for these children, Mr. Richardson developed a carrying case containing prerecorded songs and a procedure to follow which could be used by Child Life Specialists in his absence.

This technique continues to be used with patients of all ages at M. D. Anderson. Currently the technique is taught to patients to help with relaxation or change their mood. If the patient has their own music with them, the patient can organize it according to the ISO-Vectoring Principle. The automated program of an embodiment disclosed herein can and will benefit more patients, improve the exactness of the process of music selection and organization, and increase the number of patients served by music therapists. Hence, an embodiment disclosed herein is directed to a computer implemented methodology which codifies and further develops this therapy.

The computer-implemented method generally filters and sorts the music tracks according to the musical genre (selected by a user) and music tempo which is expressed in beats per minute (also referred to herein as BPM). BPM is a unit typically used as a measure of tempo in music or a measure heart rate. A rate of 60 BPM means that one beat will occur every second. One BPM is equal to 1/60 Hz.

Although BPM can be calculated by hand, existing software programs can do this automatically by listening for regular volume peaks at low frequencies. Additionally, such existing software programs are designed to tag the music tracks with a BPM label. In accordance with some embodiments disclosed herein, the computer-implemented method interfaces with preexisting software programs such as Itunes or windows-based music library programs. These programs can generally assign the BPM to music tracks in a library of such tracks. The user can organize the music manually or the program will organize the music automatically based on a pre-specified outcome that is desired.

The computer-implemented method also incorporates vectoring by further filtering the play list to allocate the music tracks to play in order of appropriate stages creating a progressive modulation of mood from the initial mood to the target mood. Each stage represents a range of speeds of music based on the BPM designations. For example, music may be ordered in five separate stages according to BPM, with a range extending from about 130 BPM down to about 40 BPM. With this range the various stages of music would correspond to 40-58 BPM, 58-76 BPM, 76-94 BPM, 94-112 BPM, and 112-140 BPM. In addition to sorting music by BPM, the user may initially select a genre of music that does an initial filtering of a master list of music tracks. A psychologist or clinician may assess a patient and determine that a broader range of music speeds may be necessary to calm a patient down as well. Additionally, such a qualified person may assess the readiness of the patient to progress from one stage to the next. In vectoring the music tempo in BPM is selected to match the initial mood of a person. Each stage is entered according to whether the person is to be relaxed by slowing the tempo of the music, or uplifted by increasing the tempo of the music.

Referring now to FIG. 7 which shows a flow chart of the computer-implemented method 100, in operation, the first task 110 is the tagging of all tracks in the user's library according to the tracks BPM. This is accomplished through the use of preexisting programs such as: Tangerine BPM, beaTunes, MixMeister BPM Analyzer, and BPM Detector Pro. These programs compute BPM automatically and save the information as files or ID tags.

Once all the music is tagged, the user needs to decide if they want to manually organize their tracks based on BPM for the desired outcome or to use the program. The program can either be fully automatic or the user can choose specific tracks within pre-filtered portions of the library for a specific desired effect. The tagged play list is retrieved at step 120 and the user may initiate a first filter of the play list by selecting a genre of music at step 130. The user samples the genre that is selected at step 140. This music can be temporarily stored to a local hard drive 150. If the user is content with the genre at step 160 the entire play list is filtered based on this genre at step 170.

Hence, as shown in FIGS. 6 and 7, the user will first choose music based on BPM to match their mood. This is accomplished at step 180 in which the user selects their initial mood. For example, if the user indicated 10 out of 10 on stress they would choose from fast tempo music to begin. If they indicated 5 out of 10 on stress they would choose from moderate tempo music to begin therapy. It will be understood by those skilled in the art that this example is merely exemplary and not limiting of, for example, the breadth of the scale on which stress may be measured can be extended beyond a 1-10 rating. Thus, if a patient is stressed, the first tracks will have high BPM. Music of progressively slower tempos is then used to move the patient to a more relaxed state.

Users will enter the stage of music that matches their mood based on their report of mood. For example, they could indicate how stressed or relaxed they are on a sliding scale. With the programmed logic, the software program will allocate music tracks to appropriate stages to allow the user to either become relaxed or less depressed/lift their spirits. This is accomplished at step 190 where the program further filters the play list based on initial mood and establishes the music tempo of stage 1. The program can also select music that matches a high tempo and stays at that stage or varies according to pre-specified logic (e.g., for exercise) or matches a low tempo and stays at that stage (for continued relaxation once relaxed). The user can also perform these choices on their own at optional step 200. In the manual mode the user chooses tracks to create a play list for their desired effect.

In the semi-automatic mode the user will work with the program at step 200 to create a play list for the desired effect. An optional verification sequence at step 210 may be incorporated to verify the mood of the patient and that the selected songs are of appropriate speed and appropriate for achieving the desired effect based on how the music makes the user feel. The program will filter the library of music based on BPM and allow the user to select any number of tracks that have BPM that match what they indicated their stress level was. With each song chosen being added to the play list at step 220. The songs the user chooses will be organized at step 230 based on BPM with the slower tracks coming after the faster tracks to effect relaxation.

Once a few tracks are chosen, the music library can be filtered at the next stage (step 240) based on tracks that match a slower BPM than the first few tracks chosen for stage 1. From this point on the process is iterative through stages 2-5 as indicated collective steps in 250, 300, 400, and 500 respectively. The user will continue choosing tracks that are progressively slower and slower until they have created a music play list of a desired length. The tracks available at each stage are filtered for the user and the user needs to simply choose the track they want. The tracks will be organized from faster to slower BPM typically for the purpose of obtaining a state of relaxation. When the play list is deemed complete at step 600, the play list can be sent to the next application at step 610, which will indicate the end 620 of the music selection sequence.

The user can also create a play list to lift their spirits when feeling down and blue. The interaction with the program would be the same as when helping them to relax, except in this case the user would first choose songs that are of a slow BPM and then progressively faster BPM until they have their play list of the desired length. The tracks at each stage are filtered for the user so they only can choose tracks appropriate for each stage of the vectoring.

There are other examples of how the user can use the program such as choosing music that is of slow BPM to continue relaxation or some other mood state, choose music that is fast BPM for exercise or other activity. In addition, the vectoring can take on different shapes, not just fast to slow BPM or slow to fast BPM. It could start slow and move to fast and then become moderate and then fast again. This may be useful for different forms of exercise.

The user can also have the option of interfacing with the program and/or having the music selection be fully automatic. Once the user indicates what they want to make the play list for (to help them relax, to lift their spirits, to help them get going, to motivate them while exercising, and others), they will indicate their mood if appropriate for that selection, and the program will automatically select tracks based on BPM to appropriately vector the user. If the user wants to modify any if the tracks, they can simply remove and add tracks. This will put them into the semi-automatic mode of the program.

An average play list length for helping people to relax, lift their spirits, or change their mood in some other way may be from about 25 minutes to about 35 minutes. The user can choose, however, to make a play list of their desired length, making it shorter or longer.

A play list for helping people to relax, lift their spirits, or change their mood in some other way may have fewer tracks at the beginning of the play list (stages 1 and 2) and more tracks near the end of the play list (stages 4 and 5). This will allow the user to have a greater amount of music matching the desired mood state at the end of the play list (e.g., slow BPM for relaxation, fast BPM to lift the spirits).

The computer-implemented method as describe above may be administered to modulate the mood of a patient undergoing cancer treatment. It has been found that a negative correlation exists between stress levels at the start of cancer treatment using an autologous tumor vaccine and cytotoxicity to allogeneic and autologous tumor cell targets. Importantly, low levels of stress at treatment onset were associated with an increased probability of treatment response (after controlling for disease severity). Recently, an association has been discovered between survival time of breast cancer patients and both SNS activity and the patients' self-reported mental health. Watson, M., et al., Influence of Psychological Response on Survival in Breast Cancer: A Population-Based Cohort Study, The Lancet 1999, 354:1331-6; Sephton, S. E., et al., Diurnal Cortisol Rhythm as a Predictor of Breast Cancer Survival, Journal of the National Cancer Institute. Decreasing distress and maintaining the functional integrity of the immune system are important in helping patients adjust to cancer treatment, recovery, post-treatment complications, and possibly metastatic growth. It is also important to examine the possible physiological mechanisms underlying the benefits of intervention programs for patients with cancer.

Studies show that psychosocial and psycho-educational interventions are beneficial adjunctive treatments for patients with cancer. See e.g., Fawzy, F. L., et al., Malignant Melanoma Effects of an Early Structured Psychiatric Intervention, Coping, and Affective State on Recurrence and Survival 6 Years Later Archives of General Psychiatry 1993, 50:681-9; Trijsburg, R. W., et al., Effects of Psychological Treatment on Cancer Patients: A Critical Review, Psychosomatic Medicine 1992, 54:489-517; Meyer, T. J., et al., Effects of Psychosocial Interventions With Adult Cancer Patients: A Meta-Analysis of Randomized Experiments, Health Psychology; Carey, M. P., et al., Etiology and Treatment of the Psychological Side Effects Associated With Cancer Chemotherapy: A Critical Review and Discussion, Psychological Bulletin 1988, 104:307-25; Andersen, B., Psychological Interventions for Cancer Patients to Enhance the Quality of Life, Journal of Consulting and Clinical Psychology 1992, 60:552-68.

These interventions increase perceived control, decrease the potential stigma associated with having a “psychiatric problem,” and provide a supportive environment where patients can discuss their thoughts, fears, and concerns. Meyerowitz, B. E., et al., Helping Patients Cope With Cancer, Oncology 1989, 3:120-9; See e.g., Baum, A., et al., Managing Stress and Managing Illness: Survival and Quality of Life in Chronic Disease, Journal of Clinical Psychology in Medical Settings 1995, 2:309-33. Psychosocial interventions have been shown to specifically decrease depression and anxiety and to increase self-esteem and active-approach coping strategies. Fawzy, F. I., et al., A Structured Psychiatric Intervention For Cancer Patients: I. Changes Over Time in Methods of Coping and Affective Disturbance, Archives of General Psychiatry 1990, 47:720-5; Helgeson, V. S., et al., Education and Peer Discussion Group Interventions and Adjustment to Breast Cancer, Archives of General Psychiatry 1999, 56:340-7; Richardson, J. L., et al., The Effect of Compliance With Treatment on Survival Among Patients With Hematologic Malignancies, Journal of Clinical Oncology 1990, 8:356-64. In addition, QOL, immune status, and in some cases, survival times have been enhanced by these programs. Fawzy, F. L., et al., Malignant Melanoma: Effects of an Early Structured Psychiatric Intervention, Coping, and Affective State on Recurrence and Survival 6 Years Later Archives of General Psychiatry 1993, 50:681-9; Richardson, J. L., et al., The Effect of Compliance With Treatment on Survival Among Patients With Hematologic Malignancies, Journal of Clinical Oncology 1990, 8:356-64; Spiegel, D., et al., Effect of Psychosocial Treatment on Survival of Patients With Metastatic Breast Cancer, Lancet 1989, ii:888-91; Fawzy, F. L., et al., A Structured Psychiatric Intervention For Cancer Patients: ll, Changes Over Time in Immunological Measures, Archives of General Psychiatry 1990, 47:729-35; Redd, W. H., et al., Behavioral Intervention For Cancer Treatment Side Effects, Journal of the National Cancer Institute 2001, 93:810-23.

Techniques of stress management that have proven helpful include progressive muscle relaxation, diaphragmatic breathing, guided imagery, social support, and meditation. Sloman, R., Relaxation and the Relief of Cancer Pain, Nursing Clinics of North America 1995, 30:697-709; Baider L, Uziely B, De-Nour A K. Progressive muscle relaxation and guided imagery in cancer patients. General Hospital Psychiatry 1994, 16:340-7; Moskowitz L. Psychological management of post surgical pain and patient adherence. Hand Clinics 1996, 12:129-37; Ross, M. C., et al., The Effects of a Short-Term Exercise Program on Movement, Pain, and Mood in the Elderly. Results of a Pilot Study, Journal of Holistic Nursing 1999, 17:139-47; Spiegel, D., Psychosocial Aspects of Breast Cancer Treatment, Seminars in Oncology 1997, 24:36-47; Wallace, K. G., Analysis of Recent Literature Concerning Relaxation and Imagery Interventions For Cancer Pain, Cancer Nursing 1997, 20:79-87; Walker, L., et al., Psychological, Clinical and Pathological Effects of Relaxation Training and Guided Imagery During Chemotherapy, British Journal of Cancer 1999, 80:262-8; Richardson, M. A., et al., Coping, Life Attitudes, and Immune Responses to Imagery and Group Support After Breast Cancer Treatment, Alternative Therapies in Health & Medicine 1997, 3:62-70; Turner-Cobb, J. M., et al., Social Support and Salivary Cortisol in Women With Metastatic Breast Cancer, Psychosomatic Medicine 2000, 62:337-45; Massion, A. O., et al., Meditation, Melatonin and Breast/Prostate Cancer: Hypothesis and Preliminary Data, Medical Hypotheses 1995, 44:39-46; Coker, K. H., Meditation and Prostate Cancer: Integrating a Mind/Body Intervention With Traditional Therapies, Seminars in Urologic Oncology 1999, 17:111-8. Participating in stress management programs prior to treatment has enabled patients to tolerate therapy with fewer reported side effects. Troesch, L. M., et al., The Influence of Guided Imagery on Chemotherapy-Related Nausea and Vomiting, Oncology Nursing Forum 1993, 20:1179-85; Syrjala, K. L., et al., Relaxation and Imagery and Cognitive-Behavioral Training Reduce Pain During Cancer Treatment: A Controlled Clinical Trial, Pain 1995, 63:189 98; Manyande, A., et al., Preoperative Rehearsal of Active Coping Imagery Influences Subjective and Hormonal Responses to Abdominal Surgery, Psychosomatic Medicine 1995, 57:177-82; Arakawa, S., Relaxation to Reduce Nausea, Vomiting, and Anxiety Induced by Chemotherapy in Japanese Patients, Cancer Nursing 1997, 20:342-9. Meditation may help decrease intrusive thoughts or the tendency to ruminate on or to avoid thoughts about stressors, a behavior that exacerbates the impact of stress if it continues for prolonged periods. Horowitz, M., et al., Impact of Events Scale: Measure of Subjective Stress, Psychosomatic Medicine 1979, 41:209-18; Devine, D., et al., The Association Between Social Support, Intrusive Thoughts and Avoidance, and Adjustment Following an Experimental Cancer Treatment, Psycho-Oncology In Press.

A recent systematic review of psychological therapies for patients with cancer examined the benefits of different psychological strategies for different outcomes. See Newell, S. A., et al., Systematic Review of Psychological Therapies for Cancer Patients: Overview and Recommendations For Future Research, Journal of the National Cancer Institute 2002, 94:558-84. In particular, interventions involving self-practice and hypnosis for managing conditioned nausea and vomiting are recommended. Further, the benefits of relaxation training and guided imagery warrant additional research, including the benefits of relaxation and guided imagery for managing general nausea, anxiety, QOL, and overall physical symptoms and music therapy to help manage anxiety.

The use of music in the oncological setting has been used to reduce pain, anxiety, and nausea. Zimmerman et al. reported a significant decrease in pain after patients listened to 30 minutes of relaxing music. Zimmerman, L., et al., Effects of Music in Patients Who Had Chronic Cancer Pain, Western Journal of Nursing Research, 11:298-309 (year). Likewise, a decrease in pain for patients who listened to 45 minutes of relaxing music or low frequency hum twice a day for three days compared to baseline pain has been observed. Beck, S. L., The Therapeutic Use of Music for Cancer-Related Pain, Oncology Nursing Forum 1991, 18:1327-37.

Music therapy has also been successfully used to affect mood and side effects of treatment. Evidence has shown that listening to music selected to relax the patient reduces anxiety. Frank, J. M., The Effects of Music Therapy and Guided Visual Imagery on Chemotherapy Induced Nausea and Vomiting, Oncology Nursing Forum 1985, 12:47-52; Sabo, C. E., et al., The Influence of Personal Message With Music on Anxiety and Side Effects Associated With Chemotherapy, Cancer Nursing 1996, 19:283-9. Specifically, in bone marrow transplant patients, increased relaxation and comfort levels has been reported. Boldt, S., The Effects of Music Therapy on Motivation, Psychological Well-Being, Physical Comfort, and Exercise Endurance of Bone Marrow Transplant Patients, Journal of Music Therapy 1996, 33:164-88. Additionally, cancer patients receiving 10 weekly sessions of music therapy combined with guided imagery have scored better on POMS and a cancer quality of life questionnaire than a wait-list control group. Burns, D. S., The Effect of the Bonny Method of Guided Imagery and Music on the Mood and Life Quality of Cancer Patients, J Music Ther 2001, 38:51-65. In fact, scores in the intervention group continued to increase after the completion of the weekly sessions. Furthermore, in another study patients participating in music listening and active music improvisation sessions not only showed increased relaxation and energy levels, but also showed an increase in saliva immunoglobulin A (slgA) and lower cortisol levels relative to the control group. Burns, S. J., et al., A Pilot Study Into the Therapeutic Effects of Music Therapy at a Cancer Help Center, Alternative Therapies in Health & Medicine 2001, 7:48-56.

### Example 1

### Monitoring Responses

### Sleep Disturbances

In evaluating the effectiveness of the music therapy treatment one may monitor psychological responses and bio-responses in the patient. One such assessment may be in measuring the patient's sleep disturbances. Sleep disturbances can be assessed using the Pittsburgh Sleep Quality Index (PSQI). Buysse, D. J., et al., Pittsburgh Sleep Quality Index: A New Instrument For Psychiatric Practice and Research, Psychiatry Research 1989, 28:193-213. The PSQI is a self-rated questionnaire that assesses quality of sleep and sleep disturbances over a 1-month period as follows.

The following questions are answered:

Only self-rated questions are scored. The PSQI has good internal (0.83) and test-retest reliability (0.65-0.85) and good validity, for distinguishing between patients with depression, disorder of initiating and maintaining sleep, disorder of somnolence, and healthy controls.

### Example 2

### Monitoring Responses

### Depression in Patients

Another valuable measure in monitoring the music therapy is assessing the level of depression in the patient. Depression can be assessed using the Centers for Epidemiological Studies-Depression measures (CES-D). Radloff, L. S., The CES-D Scale: A New Self-Report Depression Scale For Research in the General Population, Applied Psychological Measurement 1977, 1:385-401. The CES-D is a well-validated 20-item self-report measure of depression that focuses on affective components of depression. Respondents rate the frequency of the behavior or feeling using a 4-point Likert-type scale ranging from “almost never” to “almost always.” Internal consistency is high in the general population and in patient populations. It also has demonstrated adequate convergent validity with other measures of depression. Radloff, L. S., The CES-D Scale: A New Self-Report Depression Scale For Research in the General Population, Applied Psychological Measurement 1977, 1:385-401.

### Example 3

### Monitoring Responses

### Effectiveness of Treatment

Still another valuable indicator of the effectiveness of the treatment involves assessing changes in the mood of the patient. Changes in mood can be assessed using the Profile of Mood States (POMS). McNair, D. M., et al., Profile of Mood States, San Diego: Educational and Industrial Testing Service, 197111981. The POMS, commonly used in cancer research, is a mood adjective check-list containing six subscales: tension-anxiety, depression-dejection, anger-hostility, vigor, fatigue, and confusion-bewilderment. Reliabilities are good and range from 0.84 to 0.95. Eichman, W., The Eighth Mental Measurements Yearbook, New Jersey: The Gryphon Press, 1989. Alternatively patients may complete the POMS-SF test which is a shortened version (37 items) of the POMS for which convergent and discriminate validity of the six subscales and Total Mood Disturbance score has been established. Baker, F., et al., A POMS Short Form For Cancer Patients: Psychometric and Structural Evaluation, Psycho-Oncology 2002, 11:273-81. Reliability, analysis and subscale, means for short-form POMS are show on Table 1 below.

TABLE 1
Reliability analysis and subscale means for short-form POMS

| | No. of | Baker et al. | | Shacham | |
|---|---|---|---|---|---|
| POMS scale | Items | Alpha | Mean score | Alpha | Mean Score |
| Depression | 8 | 0.88 | 7.04 | 0.91 | 9.52 |
| Vigor | 6 | 0.91 | 12.78 | 0.87 | 7.50 |
| Confusion | 5 | 0.78 | 6.25 | 0.82 | 4.95 |
| Tension | 6 | 0.87 | 10.32 | 0.80 | 8.64 |
| Anger | 7 | 0.88 | 6.44 | 0.90 | 4.55 |
| Fatigue | 5 | 0.90 | 6.80 | 0.87 | 8.65 |

### Example 4

### Monitoring Responses

### Frequency of Intrusive Thoughts

For monitoring psychological responses one may assess the frequency of intrusive thoughts. Intrusive thoughts or the tendency to ruminate on or avoid thoughts about stressors, a possible mediator of the program's effects, will be measured using the Impact of Event Scale (IES), a 15-item self-report scale that assesses the two most common categories of responses to stressful events: intrusion (intrusively experienced ideas, images, feelings, or bad dreams) and avoidance (consciously recognized avoidance of certain ideas, feelings, or situations). Horowitz, M., et al., Impact of Events Scale: Measure of Subjective Stress, Psychosomatic Medicine 1979, 41:209-18. As provided immediately below, cognitive processing in terms of how effectively patients are adapting to a stressful, traumatic event are assessed. Respondents rate the frequency of the behavior or feeling as happening “not at all,” “rarely,” “sometimes,” or “often.”

TABLE 2
Revised Impact of Event Scale

| On you experienced | | | | |
|---|---|---|---|---|
| (date) (life event) | | | | |
| Below is a list of comments made by people after stressful life | | | | |
| events. Please check each item, including how frequently | | | | |
| these comments were true for you DURING THE PAST | | | | |
| SEVEN DAYS. If they did not occur during that time, please | FREQUENCY | | | |
| mark the “not at all” column. | Not at All | Rarely | Sometimes | Often |

| | |
|---|---|
| 1. | I thought about it when I didn't mean to. |
| 2. | I avoided letting myself get upset when I thought about it |
| | or was reminded of it. |
| 3. | I tried to remove it from memory. |
| 4. | I had trouble falling asleep or staying asleep, because of |
| | pictures or thoughts about it that came into my mind. |
| 5. | I had waves of strong feelings about it. |
| 6. | I had dreams about it. |
| 7. | I stayed away from reminders of it. |
| 8. | I felt as if it hadn't happened or it wasn't real. |
| 9. | I tried not to talk about it. |
| 10. | Pictures about it popped into my mind. |
| 11. | Other things kept making me think about it. |
| 12. | I was aware that I still had a lot of feelings about it, but I |
| | didn't deal with them. |
| 13. | I tried not to think about it. |
| 14. | Any reminder brought back feelings about it. |
| 15. | My feelings about it were kind of numb. |

Intrusion subset = 1, 4, 5, 6, 10, 11, 14;
avoidance subset = 2, 3, 7, 8, 9, 12, 13, 15.

### Example 5

### Monitoring Responses

### Effect of Music Therapy on Bioresponses

In addition to measuring psychological responses to the music therapy, one can also measure its effect on bio-responses. This may be accomplished, for example, by obtaining blood samples to assess immune function. Stress dysregulates a variety of immune indices, which has been found in both healthy subjects and people with cancer. Stone, A. A., et al., Stress and Humoral Immunity: A Review of the Human Studies, Advances in Neuroimmunology 1994, 4:49-56; Irwin, M., et al., Reduction of Immune Function in Life Stress and Depression, Biological Psychiatry 1990, 27:22-30; Cohen, L., et al., Mild Stress and Natural Killer Cell Activity in Healthy Men, Journal of Applied Biobehavioral Research 1993, 1:120-32; Antoni, M. H., et al., Cognitive Behavioral Stress Management Intervention Buffers Distress Responses and Elevates Immunologic Markers Following Notification of HIV-1 Seropositivity, Journal of Consulting and Clinical Psychology 1991, 59:906-15; Cohen, S., Psychological Factors and Physical Disease From the Perspective of Human Psychoneuroimmunology, Annual Review of Psychology 1996, 47:113-42; Kiecolt-Glaser, J. K., Chronic Stress and Immunity in Family Caregivers of Alzheimer's Disease Victims, Psychosomatic Medicine 1987, 49:523-35; Kiecolt-Glaser, J. K., Psychosocial Modifiers of Immunocompetence in Medical Students, Psychosomatic Medicine 1984, 46:7-14; Andersen, B. L., et al., Stress and Immune Responses After Surgical Treatment for Regional Breast Cancer, Journal of the National Cancer Institute 1998, 90:30-6; Tjemsland, L., et al., Preoperative Psychological Variables Predict Immunological Status in Patients With Operable Breast Cancer, Psycho-Oncology 1997, 6:311-20; Van der Pompe, G., et al., Psychoneuroimmunology and the Course of Breast Cancer: An Overview The Impact of Psychosocial Factors on Progression of Breast Cancer Through Immune and Endocrine Mechanisms, Psychooncology 1994, 3:271-88; Cohen, L., et al., Stress and Social Support Affect Immune Function in Cancer Patients Receiving Vaccine Treatment, Psychosomatic Medicine 2000, 62:128.

Studies with cancer patients have linked immune function including NK cell function and T-lymphocyte proliferation, to prognosis, recurrence, and survival time. Imai, K., et al., Natural Cytotoxic Activity of Peripheral-Blood Lymphocytes and Cancer Incidence: An 11-Year Follow-Up Study of the General Population, The Lancet 2001, 356:1795-9; Humphery, L. J., et al., Immunologic Responsiveness of Patients With Cancer: Relationship to Tumor Type, Stage and Prognosis, Annals of Surgery 1981, 193:574-8; Head, J. F., et al., Evaluation of Lymphocyte Immunity in Breast Cancer Patients, Breast Cancer Research and Treatment 1993, 26:77-88; Ader, R., et al., Psychoneuroimmunology: Interactions Between the Nervous System and the Immune System, Lancet 1995, Vol. 345:99-103; Fawzy, F. L., et al., Malignant Melanoma: Effects of an Early Structured Psychiatric Intervention, Coping, and Affective State on Recurrence and Survival 6 Years Later Archives of General Psychiatry 1993, 50:681-9; Garzetti, G. G., et al., Natural Killer Cell Activity in Stage I Endometrial Carcinoma: Correlation With Nuclear Grading, Myometrial Invasion, and Immunoreactivity of Proliferating Cell Nuclear Antigen, Gynecologic Oncology 1994, 55:111-4; Levy, S., et al., Prognostic Risk Assessment in Primary Breast Cancer by Behavioral and Immunological Parameters, Health Psychology 1985, 4:99-113; Levy, S., et al., Correlations of Stress Factors With Sustained Depression of Natural Killer Cell Activity and Predicted Prognosis in Patients With Breast Cancer, Journal of Clinical Oncology 1987, 5:348-53; Reynier, J., et al., An Experimental and Clinical Study of Immunocompetence and Immunostimulation in Breast Cancer, Internal Surgery 1988, 73:10-5.

### Example 6

### A Study of the Effect of Music Therapy on BMT Patients

Stress-reduction programs tailored to the cancer setting may help patients cope with the acute effects of treatment and improve quality of life (“QOL”) after treatment. Music therapy is one such program that is particularly useful for patients who have undergone a bone marrow transplant (“BMT”) where active involvement in a stress management may be more difficult due to treatment-related sequelae. In this study, we tested and examined the feasibility and usefulness of conducting music therapy program with cancer patients who have undergone a BMT. Our specific aims in the proposed study were:
Pilot-test music therapy to determine the acceptability and feasibility of conducting music therapy with cancer patients.
Conduct an initial evaluation of whether participation in the program improves patient quality of life and psychological outcomes.
Determine the feasibility of obtaining blood samples to assess immune function.

Patients with cancer undergoing BMT experience profound changes in their QOL. Adjusting to many of the psychological and physical changes associated with cancer is overwhelming, but stress management interventions have demonstrated that psychosocial and psychoeducational support not only improve mental health and QOL, but may improve immune status, pain indices, and length of survival and decrease hospital costs. We proposed to examine the feasibility of incorporating a music therapy program into the treatment trajectory for patients undergoing BMT.

### Bone Marrow Transplantation

Over the past 30 years, BMT has evolved from an experimental procedure to an established and effective treatment for a variety of malignant diseases. The equivocal evidence of the efficacy of transplantation for certain advanced cancers sparked recent controversy. Stadtmauer, E. A., et al., Conventional-Dose Chemotherapy Compared With High-Dose Chemotherapy Plus Autologous Hematopoietic Stem-Cell Transplantation for Metastatic Breast Cancer, Philadelphia Bone Marrow Transplant Group, New England Journal of Medicine 2000, 342:1069-76. However, these data were limited to autologous transplants for patients with advanced stage breast cancer. The National Cancer Institute and professional organizations urged caution in the interpretation of these recent studies until complete long-term data are available for analysis. BMT remains the preferred treatment for certain types of cancers, and involves many patients each year. In 1998 there were 17,000 allogeneic BMT and over 30,000 autologous BMT procedures performed in North America. 2000 IBMTR Annual Number of Blood and Marrow Transplantation World-Wide 1970-1998, International Bone Marrow Transplant Registry, 2000. Since 1990 autologous procedures have become more frequent than allogeneic ones. 2000 IBMTR Annual Number of Blood and Marrow Transplantation World-Wide 1970-1998, International Bone Marrow Transplant Registry, 2000.

Allogeneic BMT, the first type developed, involves the use of matched donor marrow. Autologous BMT, which involves the patients' own cells, was developed later to treat hematological, oncological, immunological, and genetic diseases. Decker, W. A., Psychosocial Considerations for Bone Marrow Transplant Recipients, Critical Care Nursing Quarterly 1995, 17:67-73; Parkman, R., Overview: Bone Marrow Transplantation in the 1990s, American Journal of Pediatric Hematology 1994, 16:3-5; Whedon, M. B., et al., Blood and Stem Cell Transplantation: Principles, Practice, and Nursing Insights, 2 ed. Boston: Jones and Bartlett, 1997. During autologous BMT, nonfunctioning bone marrow and/or malignant cells are eliminated with high-dose chemotherapy and/or radiation therapy. Patients then receive an infusion of their own bone marrow or peripheral blood stem cells in order to restore hematological and immunological function. Shivnan, J., et al., Bone Marrow Transplantation: Issues for Critical Care Nurses, AACN Clinical Issues 1996, 7:95-108, quiz 79-80.

Though BMT may be a life-saving intervention, it is both aggressive and life-threatening, and it is associated with a multitude of physical and psychological symptoms and sequelae. Patient management in transplantation involves providing complex care for severely ill and immunocompromised patients. The success of this treatment and survival for patients is related to successful control of symptoms of the transplantation. A consequence of this treatment is that patients develop a very low and at times absent white blood count. Therefore, prevention and early detection of symptoms is a high priority in the care of these patients.

### Symptoms in BMT

Symptoms, which vary by type of preparative regimen, transplantation, and type and stage of disease, have been linked with survival and quality of life. Symptoms are both physical and affective. Autologous BMT recipients face a 1-5% risk of death from infection and other complications, while five-year, disease-free survival rates for allogeneic BMT range from 70% (with chronic myelogenous leukemia (“CML”)) to 15% (with CML in blast crisis). Larson, P. J., Perceptions of the Needs of Hospitalized Patients Undergoing Bone Marrow Transplant, Cancer Practice 1995, 3:173-9; Champlin, R., et al., Bone Marrow Transplantation, Boston: Blackwell Scientific Publication, 1994; Thomas, E. D., et al., Marrow Transplantation for the Treatment of Chronic Myelogenous Leukemia, Annals of Internal Medicine 1986, 104:155-63. Complications are more common with BMT, and include graft-versus-host disease (GVHD). The incidence of GVHD varies widely depending on the type of transplant and the degree of mismatch with the donor. It can be as high as 80% in allogeneic BMT. Other complications of BMT include cardiac complications (40%) and neurological sequelae (59% to 70%). Shivnan, J., et al., Bone Marrow Transplantation: Issues for Critical Care Nurses, AACN Clinical Issues 1996, 7:95-108, quiz 79-80; Buchsel, P. C., et al., Delayed Complications of Bone Marrow Transplantation: an Update, Oncology Nursing Forum 1996, 23:1267-91.

While patients generally report high global quality of life scores after BMT, they continue to experience a variety of symptoms long after their BMT. Our pilot data were consistent with the literature that found three symptom clusters to be the most severe and prevalent: 1. Fatigue and sleep disturbance, including drowsiness; 2. Emotional distress, including fear of death, sadness, and feeling nervous; and 3. Nutrition issues, including lack of appetite, dry mouth, and nausea. Researchers have attempted to identify mediating and/or causal factors in the development and exacerbation of physical symptoms. Age, diagnosis, locus of control, self-efficacy, emotional distress (e.g., depression and anxiety), coping strategies, and social support have been examined. Results have indicated that emotional distress is the most significant predictor of the physical condition of autologous BMT recipients, while locus of control, self-efficacy, and coping styles are also important factors. Gaston-Johansson, F., et al., Pain and Psychological Distress in Patients Undergoing Autologous Bone Marrow Transplantation, Oncology Nursing Forum 1992, 19:41-8; Prieto, J. M., et al., Physical and Psychosocial Functioning of 117 Survivors of Bone Marrow Transplantation, Bone Marrow Transplantation 1996, 17:1133-42; Syrjala, K. L., et al., Evidence for a Biopsychosocial Model of Cancer Treatment-Related Pain, Pain 1995, 61:69-79. Recently, depression was found to predict overall survival of BMT patients after taking into account the medical variables. Loberiza, F. R., et al., Association of Depressive Syndrome and Early Deaths Among Patients After Stem-Cell Transplantation for Malignant Diseases, Journal of Clinical Oncology 2002, 20:2118-26.

At M. D. Anderson, in fiscal year 2000 a total of 548 transplants were performed, in 1999 648 were performed and in 1998, 552 were performed. These were nearly equally divided with 273 autologous transplants and 275 allogeneic in 2000, 330 autologous and 318 allogeneic in 1999, and 299 autologous and 253 allogeneic in 1998. In 2001 we anticipate performing 550 transplants. Transplants were performed for a variety of diagnoses, including leukemia (chronic lymphocytic leukemia (“CLL”) and acute myeloid leukemia (“AML”)), lymphoma, ovarian cancer, breast cancer, and others. Transplants were also performed for some non-cancer diagnoses, but such patients were not included in this study.

### Music Therapy

Music therapy has proven to be useful in a number of medical settings. Anxiety and pain control have been the main outcomes studied, A meta-analysis of randomized control studies using music as an intervention for hospital patients concludes that music is effective for the reduction of anxiety during normal care delivery. Evans, D., The Effectiveness of Music as an Intervention for Hospital Patients: A Systematic Review, Journal of Advanced Nursing 2002, 37:8-18. This analysis included 6 studies in which patients with a variety of ailments listened to music via headphones. Other studies report reductions in anxiety and pain in patients awaiting medical procedures after listening to a selection of music. Davis, C. A., The Effects of Music and Basic Relaxation Instruction on Pain and Anxiety of Women Undergoing In-Office Gynecological Procedures, Journal of Music Therapy 1992, 29:202-16; Haun, M., et al., Effect of Music on Anxiety of Women Awaiting Breast Biopsy, Behavioral Medicine 2001, 27:127-32. Additionally, less physician-administered sedation was needed by patients who listened to music during colonoscopy. Smolen, D., et al., The Effect of Self-Selected Music During Colonoscopy on Anxiety, Heart Rate, and Blood Pressure, Applied Nursing Research 2002, 15:126-36.

Although there are a few studies suggesting that music therapy may be useful in an oncology setting, there are several limitations to this research. Most of the studies have used small samples, and only a few used a randomized, controlled design. Many interventions also had the participants listen to music from a pre-determined selection. It is not clear, therefore, what role if any an actual music therapist plays in the process. Guided imagery was also frequently combined with the music, and therefore it is hard to determine which component is affecting a change.

### ISO Principle

In developing a music therapy program to administer in a research setting aspects of ease of administration and reproducibility are important constructs. As described above, there are a variety of forms music therapy can take. We examined the use of the ISO principle using individualized tailored audio CDs.

### Research Overview and Design

This study evaluated the feasibility and explored the impact of a 4-session integrated music therapy intervention using a randomized-controlled experimental design with repeated measures, with two experimental treatment conditions and one control group. Patients who have undergone a BMT and have transitioned to outpatient status during the first 100 days post-transplant are recruited to participate. Patients were identified through the CARES system and through members of the BMT team, and approached at the end of their inpatient stay or at the ambulatory treatment center (ATC) where they are receiving their care as an outpatient.

After describing the study, the willingness of the patients to participate was determined by the research coordinator and eligible patients are recruited. After obtaining informed consent from the patients, an initial assessment and initial measures were obtained. Following this, the patients are assigned to the music therapy (MT) group, the relaxing music (RM) group, or the usual care (UC) control group. The RM group will control for therapist contact and the general benefits of listening to music to help modify mood. We determined whether the aspect of the ISO principle is beneficial above and beyond simply listening to relaxing or uplifting music. A future larger study could further examine how much the music therapist contributes and whether having a choice over the music is beneficial. Patients were informed that we are examining the benefits of different forms of music therapy. They were not told the specific details of the two groups in order not to bias the outcomes. They were not told that if they are assigned to one of the music therapy groups, they will work with a music therapist to select specific music that may help them relax and specific music that may help lift their spirits.

Participants in the treatment groups received four sessions, each lasting about 50 minutes, which take place at the MDACC. During the first two sessions, participants selected music to make two audio CDs. During sessions 3 and 4, they reviewed the material. They are given a portable CD player and the two tailored CDs to take home and use at home and when they come to MDACC. Patients completed a brief assessment before and after using the CDs at the fourth session. Further assessments of the participants were at one week, one month, and three months intervals after the last session. The first two follow-up assessments were during the first 100 days, and the 3-month follow-up assessment corresponds approximately with the 1 month visit after the first 100 days. The patients who were assigned to the UC group have their assessments at a comparable point in time as the two intervention groups. Patients in the UC group were given the option of receiving two audio CDs after the 3-month follow-up assessment.

If the patients are not coming for a follow up visit or are unable to travel, a questionnaire was mailed to them.

Patient Eligibility/Exclusion/Removal from Study Criteria

### Inclusion Criteria

Patients who have undergone an allogeneic BMT at the M. D. Anderson Cancer Center were eligible to participate in the study. Patients needed to be about to transition to an outpatient status or are already an outpatient during the first 100 days from transplant. The participants need to be 18 years of age or above, since the assessment tools are not validated for minors. They had to be able to read, write, and speak English.

### Exclusion Criteria

Patients who have a known psychotic disorder were excluded from the study, in order to ensure proper assessment of the efficacy of intervention program.

Study Population

Participants were patients who have undergone an allogeneic BMT at M. D. Anderson and were transitioning from inpatient status to outpatient status. Seventy-five patients were recruited. Based on our previous research, we expected a 15% rate of attrition. All patients were recruited through the BMT service at M. D. Anderson.

### Study Protocol and Procedures

### Recruitment

Potential study participants were identified through the CARES database and by the BMT physicians. Eligible patients were approached by a research assistant while they are an inpatient, or during the transition period from inpatient to outpatient, or when they are being followed as an outpatient at the ATC. The purpose of the initial contact was for the research assistant to describe the study in detail, to answer patient questions, and to assess patient interest in the study. Information to determine eligibility for the study and basic demographic information was collected from all patients contacted regarding the study. Patients who meet the inclusion/exclusion criteria and express an interest in the study scheduled for the baseline assessment.

### Procedures

If consent has not already been obtained, informed consent was collected when the patient arrives for the baseline assessment. The baseline assessment includes a 30-minute battery of questionnaires. Patients were randomized following completion of all baseline questionnaires. Patients also provided a 40-ml blood sample at baseline. After patients are randomized, they were scheduled for the first music session. The same measures were collected 1 week, 1 month, and 3 months after the completion of the music therapy sessions.

Participants in the two music groups participated in four music sessions. The sessions took place at the M. D. Anderson in the ATC. The sessions for the MT group were conducted by a board certified music therapist who has been working at M. D. Anderson for the past 5 years. The sessions for the RM group were conducted by a board certified music therapist or licensed counselor/therapist. The participants completed the POMS-SF at the start and end of the last session. All major assessments (baseline, and 1 week, 1 month, and 3 months) were conducted by research assistants or other project members who are blind to group assignment. The patients who are assigned to the UC group were given the option of receiving some relaxing music CDs at the end of the study. Patients are given a small gift certificate (a $20 value) after completing each of the evaluations at baseline, and 1 week, 1 month, and 3 months after the end of the sessions.

### Group Assignment

After the participants were recruited and initial measures were obtained, patients were assigned to either the MT, RM, or UC groups by a form of adaptive randomization, minimization, because this is a small study and simple randomization could result in covariate imbalances. Pocock, S. J., Clinical Trials: A Practical Approach, New York: John Wiley & Sons, 1983. Statistical adjustment of covariates can take imbalances into consideration, but results are generally more credible when they are obtained from groups with comparable baseline distributions. In minimization, group assignment is done sequentially. Before a participant is assigned to a treatment group, the number of already randomized participants with similar covariate characteristics is totaled. The totals are computed based on marginal sums so that each covariate is considered separately. The treatment assignment for a participant is then based on which treatment group assignment would produce the best overall balance with respect to the covariate characteristics. Minimization is similar to stratification in that participant characteristics are used to assign participants to the treatment conditions. Minimization, however, results in better group balance and does not suffer from the limitations of stratification, such as the increased probability of group imbalance when several participant factors are used. In this study, the patient characteristics used for group assignment are the status at the time of transplant (remission versus not in remission), cellular source, age, time since initial diagnosis, baseline anxiety score, and type of transplant procedure (mini versus traditional).

### Music Program

The sessions for the two music treatment groups are provided similar in format. The main difference is that the MT group is introduced to and chose their music based on the ISO principle and the RM music group is introduced to using music to modify mood and they choose music to help them relax and feel less depressed. Both groups spend time discussing the use of music to help them relax and feel better, they spent time listening to and choosing music, and they are provided a “prescription” for when to use the CDs. Both groups are provided two CDs. They are encouraged to use one of them to help them relax and the other to help them feel less depressed. Both groups participate in four sessions lasting 50 minutes each. At the end of Session 3, the participants in both groups are provided with a portable CD player and the two CDs they made. They returned the CD players at the 3-month follow-up assessment and they are able to keep their CDs.

### MT Group

The main focus of the MT group is to introduce the participants to the ISO principle, work with them to make two audio CDs (one to relax and the other to feel less depressed), and provided them a prescription for the use of the CDs.

### Session 1 and 2

Session 1 focuses on introducing the ISO principle and reviewing the use of music to help patients relax and feel less depressed. During the remainder of Session 1 they work with the therapist to choose songs (tracks) for their first CD “Music to Help You Relax.” This is accomplished by having them listen to different tracks for 30-60 seconds until they identify 15 tracks that they like. The therapist guides the participants to tracks that fit within the five different stages of the ISO principle. The stages are defined based on tempo and musical content (e.g., words and types of instrumentation). The objective is to have 2 tracks for each of the 5 stages for a total of 10 tracks. This corresponds to approximately 30 minutes of music. Because of the varying length of music the total time may vary somewhat, but we restrict it to be between 25 and 35 minutes worth of music. Session 2 focuses on finishing the choice of tracks for the first CD and choosing music for the second CD “Music to Lift Your Spirits.” Before the start of Session 3, the therapist burns the two CDs ensuring that the order corresponds to the ISO principle.

### Session 3

During Session 3, the participants review both CDs with the therapist. If changes need to be made, they have an additional five tracks for each of the CDs from which they can choose substitutes.

### Session 4

At Session 4, the participants choose one of the CDs and listen to the whole CD. They complete a brief mood questionnaire before and after listening to the CD. After listening to the CD, they review their thoughts with the therapist. At the conclusion of the session the therapist discusses the different uses for the CDs and provides a written description describing the different times when using the CDs may be useful.

### RM Group

The main focus of the RM group is to introduce the participants to the use of music to modify mood, work with them to make two audio CDs (one to relax and the other to feel less depressed), and provide them a prescription for the use of the CDs.

### Session 1 and 2

Session 1 focuses on introducing them to the use of music to modify mood, particularly to help patients relax and feel less depressed. During the remainder of Session 1 they work with the assistant to choose songs (tracks) for their first CD “Music to Help You Relax.” This is accomplished by having them listen to different tracks for 30-60 seconds until they identify 15 tracks that they think will help them feel relaxed. The objective is to have a total of 10 tracks. This corresponds to approximately 30 minutes of music. Because of the varying length of music the total time may vary somewhat, but we restrict it to be between 25 and 35 minutes worth of music. Session 2 focuses on finishing the choice of tracks for the first CD and choosing music for the second CD “Music to Lift Your Spirits.” Before the start of Session 3, two CDs are burned in the order of the patients' preference and available for review at Session 3.

### Session 3

During Session 3, the participants review both CDs with the therapist. If changes need to be made, they have an additional five tracks for each of the CDs from which they can choose substitutes.

### Session 4

At Session 4, the participants choose one of the CDs and listen to the whole CD. They complete a brief mood questionnaire before and after listening to the CD. After listening to the CD they review their thoughts with the therapist. At the conclusion of the session the assistant discusses the different uses for the CDs and provide a written description describing the different times when using the CDs may be useful.

### Research Materials Obtained

Several measures we used previously in our work on QOL and stress was used in this study. All of these measures have proved useful and sensitive and are also stable, reliable, and valid when used for groups of people who are not suffering from major depression or other psychiatric disorders. As described in some instances above, multimodal, simultaneous assessments provide a more complete and informative picture of QOL and responses to stress.

These Materials Collected to Meet Specific Aim 1: Determine the Acceptability and Feasibility of Conducting Music Therapy with Patients with Cancer.

Patient satisfaction with the intervention is assessed at 1 week, 1 month, and 3 months after the music therapy session. Participants are asked to identify aspects of the program they liked or disliked and identify what they found most and least useful. The frequency is assessed in addition to a Listening Log they keep.

Patients' experience with participating in the study and having cancer is assessed qualitatively by asking patients to write for 20 minutes about their experiences participating in the study, their feelings about being diagnosed with cancer, and the effect of the cancer on their present and future. The writing is conducted at the 1-week post intervention assessment.

Tracking data is kept on patient interest during the recruitment period, study attrition, and completion of questionnaires.

### The Following Materials Collected to Meet Specific Aim 2: Conduct an Initial Evaluation of Whether Participation in the Music Program Improves Patients' QOL and Objective Physiological Outcomes.

This study is primarily a feasibility study, and although we do not have the power needed to detect small or even moderate treatment group differences for all of these measures, investigators are able to assess the return rate and completion of the questionnaires. These are helpful for several reasons. One is that these determine whether the measures, the length of assessment, and the number of assessments are acceptable to patients. This is important because these measures and follow-up assessments would be performed in a larger study with adequate power to detect changes in the measures. In addition, scores obtained from the measures are useful for generating group means, effect estimates, and confidence intervals that can be used in future studies for determining sample size and power. Finally, we might be able to detect a difference between the control and treatment groups' outcome measures; however, any detectable difference would need to be large because on the small sample. All participants complete these measures at four time points over the course of the study. For members of the treatment groups, those time points are at entry into the study and 1 week, 1 month, and 3 months after completing the program. Participants in the control group complete the measures at entry into the study and at corresponding points in time relative to the treatment groups.

The Functional Assessment of Cancer Therapy-Bone Marrow Transplant (FACT-BMT) is a cancer-specific measure of health-related QOL. It is essentially the FACT-G, a general measure of QOL in patients with cancer, with an additional subscale designed to assess QOL issues particularly relevant to patients who have undergone a BMT. Cella, D. F., et al., The Functional Assessment of Cancer Therapy Scale: Development and Validation of the General Measure, Journal of Clinical Oncology 1993, 11:570-9. This measure yields an overall QOL score and five subscales: physical well-being, social or family well-being, emotional well-being, functional well-being, and treatment-specific concerns. The scale has been found to have good concurrent validity, high internal consistency (alpha=0.89), and good test-retest reliability (0.82-0.88).

Cancer-related symptoms are assessed using the M. D. Anderson Symptom Inventory (MDASI). As shown in FIGS. 8 and 10, the MDASI consists of a core list of symptoms that are common across all cancer diagnoses and treatments, plus modules of additional symptoms that can be included for patients who are receiving aggressive treatments. The results of a recent study of over 500 outpatients with cancer who completed the MDASI supported the reliability and validity of the instrument. Cleeland, C. S., et al., Assessing Symptom Distress in Cancer: The M. D. Anderson Symptom Inventory, Cancer 2000, 89:1634-46. Patients rate the intensity of physical, affective, and cognitive symptoms on 0 to 10 numeric scales from “not present” to “as bad as you can imagine.” Patients also rate the amount of interference with daily activities caused by symptoms on 0 to 10 numeric scales from “did not interfere” to “interfered completely.”

As shown in FIGS. 9 and 11, fatigue is assessed using the Brief Fatigue Inventory (BFI). Mendoza, T. R., et al., The Rapid Assessment of Fatigue Severity in Cancer Patients, Cancer 1999, 85:1186-96. The BFI is a 9-item questionnaire designed to be used in the clinical setting to rapidly assess fatigue severity. The items are ranked from 0 to 10, and patients rate their fatigue at its “worst” and “usual” and as it is “now,” with 0=“no fatigue” and 10=“fatigue as bad as you can imagine.” Patients also rate how much their fatigue has interfered with their life. This single-dimension instrument was tested in a sample of 305 patients with cancer, and provided an internally stable measure of fatigue severity (0.80-092). Mendoza, T. R., et al., The Rapid Assessment of Fatigue Severity in Cancer Patients, Cancer 1999, 85:1186-96.

Sleep disturbances are assessed using the Pittsburgh Sleep Quality Index (PSQI). Buysse, D. J., et al., Pittsburgh Sleep Quality Index: A New Instrument For Psychiatric Practice and Research, Psychiatry Research 1989, 28:193-213. The PSQI is an 18-item self-rated questionnaire that assesses quality of sleep and sleep disturbances over a 1-month period. A total score is derived as well as seven subscales including subjective sleep quality, sleep latency, sleep duration, habitual sleep efficiency, sleep disturbances, use of sleeping medications, and daytime dysfunction. The instrument includes some open-ended questions, such as asking the respondent's usual bedtime and usual number of hours of sleep. Other items ask respondents to indicate frequency of sleep disturbances as “not during the past month,” “less than once a week,” “once or twice a week,” or “three or more times a week.” The PSQI has good internal (0.83) and test-retest reliability (0.65-0.85). The PSQI was found to have good validity, and for distinguishing between patients with depression, disorder of initiating and maintaining sleep, disorder of somnolence, and healthy controls. FIG. 12 depicts data for sleep disturbances one month after post treatment.

Depression is assessed using the Centers for Epidemiological Studies-Depression measures (CES-D). Radloff, L. S., The CES-D Scale: A New Self-Report Depression Scale For Research in the General Population, Applied Psychological Measurement 1977, 1:385-401. The CES-D is a well-validated 20-item self-report measure of depression that focuses on affective components of depression. Respondents rate the frequency of the behavior or feeling using a 4-point Likert-type scale ranging from “almost never” to “almost always.” Internal consistency is high in the general population and in patient populations. It also has demonstrated adequate convergent validity with other measures of depression. Radloff, L. S., The CES-D Scale: A New Self-Report Depression Scale For Research in the General Population, Applied Psychological Measurement 1977, 1:385-401. FIG. 13 depicts depression data one month after post treatment.

Changes in mood are assessed using the Profile of Mood States (POMS). McNair, D. M., et al., Profile of Mood States, San Diego: Educational and Industrial Testing Service, 197111981. The POMS, commonly used in cancer research, is a mood adjective check-list containing six subscales: tension-anxiety, depression-dejection, anger-hostility, vigor, fatigue, and confusion-bewilderment. Reliabilities are good and range from 0.84 to 0.95. Eichman, W., The Eighth Mental Measurements Yearbook, New Jersey: The Gryphon Press, 1989. Participants complete the POMS-SF which is a shortened version (37 items) of the POMS for which convergent and discriminate validity of the six subscales and Total Mood Disturbance score has been established. Baker, F., et al., A POMS Short Form For Cancer Patients Psychometric and Structural Evaluation, Psycho-Oncology 2002, 11:273-81. FIG. 14 changes in mood data one month after post treatment.

Intrusive thoughts or the tendency to ruminate on or avoid thoughts about stressors, a possible mediator of the program's effects, are measured using the Impact of Event Scale (IES), a 15-item self-report scale that assesses the two most common categories of responses to stressful events: intrusion (intrusively experienced ideas, images, feelings, or bad dreams) and avoidance (consciously recognized avoidance of certain ideas, feelings, or situations). Horowitz, M., et al., Impact of Events Scale: Measure of Subjective Stress, Psychosomatic Medicine 1979, 41:209-18. This scale assesses cognitive processing in terms of how effectively patients are adapting to a stressful, traumatic event. Respondents rate the frequency of the behavior or feeling as happening “not at all,” “rarely,” “sometimes,” or “often.” A recent study of 80 women with newly diagnosed breast cancer showed that the instrument had good internal consistency at three separate assessments in terms of the intrusion and avoidance subscales (0.70 to 0.85). Epping-Jordan, J. E., et al., Psychological Adjustment in Breast Cancer: Processes of Emotional Distress, Health Psychology 1999, 18:315-26. The correlation between the scales was modest, however, suggesting that the scales represent relatively distinct constructs. The scale is generic in design but for this study, patients are asked to rate the frequency of intrusive thoughts and avoidance behaviors related to their cancer.

### Medical Markers, Treatment and Risk Factors

Staging information; time since diagnosis; treatment regimen; risk factors such as age, family history, and ethnicity; and other background information is obtained from medical records. These variables are entered into analyses as covariates. Medical complications are recorded.

### Immune Measures

These materials are collected to meet the Specific Aim 1.3: To determine the feasibility of obtaining blood samples to assess immune function.

Blood samples are obtained at baseline, one week, one month, and three months after intervention sessions. Blood samples are collected in heparinized vacutainer tubes (40 ml total). All immune assays PBMCs are separated using Ficoll-Hypaque gradient centrifugation, washed twice, and resuspended in RPMI-complete medium. An automated hematology analyzer determines cell counts, and viability is determined by Tryptan Blue dye exclusion criteria. The cells are then suspended to a concentration of 5-10×106/ml in a solution of 90% human AB serum and 10% dimethylsulfoxide, and cryopreserved. Our experience has shown that cells preserved in this fashion maintain functional activity and phenotypic characteristics as tested in the assays described below. Thawing and testing of the specimens from all time points simultaneously on the same day is not only economically more efficient, but eliminates the day-to-day variability inherent in many of the assays.

The immune measures assessed in this study include cytotoxicity to K562 target cells; stimulated release of IL-2, IFN, IL-4, and IL-10; flow cytometric analysis of intracellular cytokines (to identify the subset of cells producing the particular cytokine); phenotype (CD3, CD56, CD16, CD4, CD8, Ki-67, CD45 RA, and CCR7 (homing molecule); thymic function is assessed by quantitating the number of recent TRECs formed during TCR chain rearrangement using a modification of the PCR-based method; assessment of T cell activation by flow cytometry using FITC-, PE-, PerCP-, and APC-conjugated monoclonal antibodies (MAb) specific for human CD4, CD8, CD69, anti-IFN. Other assays may be conducted in the future.

### Potential Risks and Procedures for Protection Against Risks

Completing the Questionnaires: Some questions may be sensitive, and patients may refuse to answer any questions that make them feel uncomfortable. However, the completion of questionnaires usually does not cause patients to experience distress. In the unlikely event that psychological problems or questions are triggered by participation in this study, counseling is provided at no cost.

Psychiatric and psychological support is available through the Department of Neuro-oncology. In the event that patients report persisting distress, suicidal ideation, or they simply report difficulty in coping with treatment, side effects or other issues, appropriate referrals are made. The data collected includes information on any psychiatric care and counseling obtained by patients outside the study.

### Statistical Considerations

Analyses of the pilot study data is conducted in two phases to reflect the hypotheses, the first being the descriptive feasibility and process evaluation and the second being the outcome analyses.

### Feasibility and Process Evaluation

We examine our records of the patients' reported satisfaction with the intervention. This is primarily descriptive in nature including the percent of patients who completed the program, patients' ratings of the various aspects of the intervention program, acceptability of the set of exercises, perceptions about the benefits of those exercises, and other similar descriptors. The writings by the patient of her perceptions about having cancer are assessed in an exploratory fashion and focuses on the qualitative analysis, including the use of ethnographic methods. This is accomplished using NUD*IST and LIWC, software designed to analyze open-ended interviews. NUD*IST is used to summarize text into groups of statements or phrases of similar content. LIWC or Linguistic Inquiry and Word Count was developed by Pennebaker and colleagues to examine the linguistic content of writing. Pennebaker, J. W., et al., Cognitive, Emotional, and Language Processes in Writing: Health and Adjustment to College, Cognition and Emotion 1996. This software counts the number of times specific words have been used in an essay. The software recognizes more than 2,290 words and classifies them into 74 different grammatical, cognitive, and emotional categories.

Other more formal process evaluation methods can also be employed, including assessing the potential internal and external validity of the study results. To assess the external validity of the study, the demographic information on both participants and non-participants are compared using t-tests and chi-squared tests of homogeneity. Two dichotomous endpoints can also be constructed to conduct multivariate logistic regression analyses. The first denotes whether an eligible subject was successfully recruited, and the second indicates whether a participant was lost to follow-up. Predictor variables include demographic characteristics, disease information, and baseline QOL (for loss to follow-up). Stepwise regression, with a rejection criterion of p>010, is used to build the final models. This information helps determine the characteristics of patients recruited into the study and retained for the study duration. This information helps guide future recruitment efforts and identifies the types of patients who may require special attention to minimize attrition. To assess the internal validity of the study results, the percent of patients who drop out from each of the three study groups can be compared at each follow-up time point using chi-squared tests of homogeneity. The demographic, medical, and psychosocial characteristics collected at the baseline assessment of both patients who drop out and those who do not can be compared using t-tests and chi-squared tests of homogeneity.

Potential moderation of the treatment effect can be determined by evaluating the extent to which the planned intervention components were actually delivered and whether study members participated in other similar programs or interventions. This information can be obtained by reviewing the exposure tracking database, the implementation checklists of activities, and the participant interviews. Relevant information includes the level of session attendance by the treatment groups, how much the treatment group members used the CDs outside of the classes, and the degree of participation in stress-reduction programs outside of this study.

### Outcome Analyses:

Before inferential procedures begin, extensive descriptive analyses is conducted for each QOL outcome measure at each of the four time points (baseline and 1 week, 1 month, and 3 months after the program has ended) for each study group. Descriptive statistics (e.g., means, ranges, and standard deviations) are computed for the measures, together with 95% confidence intervals for the means. Graphic methods (e.g., box plots and histograms) can be employed to closely examine distributions of the measures at each time point. Bivariate associations between the outcome measures and selected demographic and disease-related variables, including age, ethnicity, time since diagnosis, and disease stage, can be evaluated using Pearson's product-moment correlation coefficients, together with scatter plots where appropriate.

The outcomes (QOL) recorded in this study are repeated measurements on multiple outcomes, possibly including hierarchical structure over subscales and related outcome measures. Desired inference includes hypotheses about treatment effects at different times and on different outcomes. Also, inference about treatment effects has to appropriately adjust for recorded baseline characteristics of the enrolled patients. With these considerations in mind, we proposed using generalized linear mixed model regression (GLMM). Separate sets of analyses will be conducted for each criterion variable. For each criterion variable, we will use data across the set of post-intervention assessment points. In modeling these data, intervention condition is a between-subjects factor, time is a within-subjects factor, and baseline measures will be included as covariates.

Generalized linear mixed modeling is a flexible analytic approach with wide use in the health sciences. Mixed model regression allows for repeated measures across individuals by modeling the correlation among the repeated measures. GLMM can accommodate a range of correlation structures among the measures, as well as continuous and discrete outcome distributions, unbalanced designs, different link and variance functions, and, in the case of the analysis of independent data, is equivalent to logistic, ordinary least squares regression, or analysis of covariance modeling.

### Sample Size Calculations

Although this is primarily a feasibility study, descriptive statistics used to characterize participants' characteristics and responses to survey items using means and percentages. Using a power approach to planning a single-factor analysis, it is determined that a sample of size 21 patients for each treatment group is required to detect a treatment effect equal to one unit of standard deviation at a 5% significance level with 80% power. Anticipating a 16% attrition rate during the study, 90 participants were recruited, distributing the patients equally among the three treatment groups. Within each treatment condition, a sample size of 21 subjects allows us to estimate percentages to within 21 percentage points and means to within 0.46 standard deviation units with 95% confidence.

### Data Management and Quality Control

Participant responses are coded, entered in the computer and managed by a data manager. For quality assurance, the data manager practices two-step data entry with first entry soon after the data collection and the second. The in-house computer network allows online entry and editing.

### Results of Initial Study

In its original formulation, the ISO technique for psychiatric patients was to match the mood or mental tempo of the patient and use rhythm, melody, mood-modifying music, harmony or pictorial-associative music to alter psychological state such as hallucinations, illusions, fears and the like. We observed that a variation of the current ISO-Vectoring Principle on the inpatient pediatric unit is useful to help children who were undergoing bone-marrow aspirations and lumbar punctures. The focus is to help decrease pain and anxiety and create a calming environment. Our use of the ISO-Vectoring Principle is unique and different because music was selected based on patient preference and organized to meet current (1993) definitions of the “ISO-Vectoring Principle” or as some therapists called it “The iso-Moodic Principle.”

As noted, the above experimental protocol was developed to examine the benefits of the ISO-Vectoring Principle for patients who had recently undergone a bone-marrow transplantation (BMT). We conclude that music therapy is particularly useful for patients who have undergone a BMT where active involvement in a stress management may be more difficult due to treatment-related sequelae. In this study, we pilot-tested and examined the feasibility and usefulness of conducting music therapy program with cancer patients who have undergone a BMT. We evaluated a 4-session integrated music therapy intervention using a randomized-controlled experimental design with repeated measures, with two experimental treatment conditions and one control group. Patients who had undergone a BMT and had transitioned to outpatient status during the first 100 days post-transplant were recruited to participate.

After collecting baseline measures patients were assigned to a music therapy (MT) group, a relaxing music (RM) group, or a usual care (UC) control group. Patients in the MT group worked with Mr. Richardson to develop two personalized CD's to “help them relax” and to “lift their spirits” following the ISO-Vectoring Principle. Patients in the RM group worked with a therapist to develop personalized CD's but simply chose songs that they thought would help them relax or lift their spirits. Patients in the UC group did not meet with a therapist. All patients completed self-report measures about their quality of life and mental health at baseline and then again 1 week and 1 month after the completion of the music therapy sessions.

Initial analyses of the data have been completed. Ninety patients were randomized one of the three groups (MT=29; RM=30; UC=31). Although there were no statistically significant differences between the three groups, all the means were in the expected direction. For example, by the 1 month follow-up assessment the patients in the MT group reported the lowest levels of fatigue (means adjusted for baseline: MT=2.5, RM=3.0, UC=3.2), cancer related symptoms (means adjusted for baseline: MT=35, RM=39, UC=43), sleep disturbances (means adjusted for baseline: MT=7.0, RM=9.1, UC=8.5), fewer depressive symptoms (means adjusted for baseline: MT=8.8, RM=9.6, UC=9.5), and lower levels of mood disturbance (means adjusted for baseline: MT=6.3, RM=6.8, UC=7.4). Patients in the MT group also made comments such as “must have been extremely relaxed, I fell asleep, I felt peaceful”, “helped relax for sleep” “it is calming” and “anxious about tests at M. D. Anderson.” Music helps me unwind” and be “less anxious.”

Disclosed herein is a computer-implemented method of organizing music tracks with a user interface comprising providing for a display of program types, wherein the program types comprise an automatic program, a semi-automatic program, a manual program, an exercise program, and a therapy program, providing for selection of one of the program types by a user interface means, and providing for instruction to execute the selected program type, wherein the music tracks are played in an organized manner according to user requirements and objectives.

Disclosed herein is a computer-readable medium containing instructions for controlling a computer system with a user interface to carry out a method of organizing music tracks, the method comprising the steps of providing for a display of program types, wherein the program types comprise an automatic program, a semi-automatic program, a manual program, an exercise program, and a therapy program, and providing for selection of one of the program types by a user interface means, wherein the music tracks are played in an organized manner according to user requirements and objectives.

Disclosed herein is a program storage device readable by a computer equipped with a user interface, embodying a program of instructions executable by the computer to perform the steps for organizing music tracks wherein the program comprises the steps of providing for a display of program types, wherein the program types comprise an automatic program, a semi-automatic program, a manual program, an exercise program, and a therapy program, and providing for selection of one of the program types by a user interface means, wherein the music tracks are played in an organized manner according to user requirements and objectives.

Disclosed herein is a computer-implemented method of sorting music tracks based on the beats per minute of the music (BPM/tempo) and using the sorted music tracks to modulate the mood of a person. For example, if the person is depressed the music may begin at a slow tempo and increase in speed stepwise to uplift the spirit of the person. Alternatively, if the person is experiencing panic, anxiety or the like, the music may begin at a more rapid tempo and decrease in speed stepwise to relax the person. The method may also be used as a means of mood maintenance, when a person is generally in a desirable mental state. Mood, as used herein, includes, but is not limited to mental states of depression, stress, tension, panic, anxiety, fear, contentedness, happiness, well-being, relaxation, and generally uplifted spirit.

The computer-implemented method of organizing music tracks with a user interface may be stored on computer-readable medium containing instructions for controlling a computer system to carry out the method. The program storage device readable by a computer, embodying the computer-implemented method may be a disk or a hard drive, for example.

FIG. 15 shows a flow chart of computer-implemented method of organizing music tracks with a user interface. As shown in FIG. 15, the user will choose a program type selected from the group consisting of: an automatic program, a semi-automatic program, a manual program, an exercise program, and a therapy program. Once the user has selected one of the program types by a user interface means, the computer-implemented method will then execute the selected program type. The music tracks are then played in an organized manner according to user requirements and objectives. User interface means may be a keyboard, voice recognition, a touch screen, or any other user interface mechanism known in the art.

The automatic program comprises the steps of choosing a vector pattern from a plurality of pre-defined patterns based on BPM, providing for retrieval of available music tracks to fit the vector pattern, providing for automatic assignment of music tracks to said music pattern; and providing for instruction to play the music tracks. FIG. 16 shows a flow chart of the semi-automatic program.

The semi-automatic program comprises the steps of choosing a vector pattern from a plurality of pre-defined patterns based on BPM, providing for retrieval of available music tracks to fit the vector pattern, providing for the user to choose said music tracks, and providing for instruction to play the music tracks. FIG. 17 shows a flow chart of the semi-automatic program.

The manual play program comprises the steps of providing for retrieval of available music tracks based on BPM, providing for addition of music tracks to a play list, providing for inquiry of completion of the play list, and providing for instruction to play the play list. FIG. 18 shows a flow chart of the custom play program.

The exercise program comprises the steps of providing for retrieval of available music tracks, providing for selection of a routine type, wherein the routine type is chosen from a custom routine and a plurality of pre-defined routines, wherein the custom routine comprises providing for addition of music tracks to a play list based on BPM, providing for inquiry of completion of the play list, and providing instruction to play the play list, and wherein the plurality of pre-defined routine filters music tracks based on BPM and comprises providing for a warm up routine, providing for a running routine, and providing for a high impact routine, and providing for execution of the selected routine. These are just examples. FIG. 19 shows a flow chart of the exercise program.

The therapy program comprises the steps of providing for selection of an appropriate target mood, providing for instruction to filter a play list of music tracks, and providing for instruction to vector by further filtering the play list to allocate the music tracks to play in order of appropriate stages creating a progressive modulation of mood from an initial mood to the target mood, wherein the music tracks are sorted by music tempo in beats per minute. FIG. 7 shows a flow chart of the therapy program.

The therapy program can be executed in one of three modes: 1) fully automatic where the music tracks are chosen by the program to fit a plurality of pre-defined vector patterns, 2) semi-automatic where the user will choose the music tracks to fit a plurality of pre-defined vector patterns, and 3) manual where the user will choose the vector pattern and the music tracks.

While specific embodiments disclosed herein have been shown and described in detail to illustrate the application of the principles disclosed herein, it will be understood that the principles disclosed herein may be embodied otherwise without departing from such principles.

What is claimed is:

1. A computer-implemented method of modulating a mood of a person comprising the steps of:
selecting an appropriate target mood;
filtering a play list of music tracks; and
vectoring by further filtering the play list to allocate the music tracks to play in order of appropriate stages creating a progressive modulation the mood from an initial mood to the target mood, wherein said music tracks are sorted by music tempo in beats per minute.

2. The method of claim 1, wherein the target mood is selected from the group consisting of: relaxed, uplifted, and same.

3. A computer-implemented method of modulating a selected state in a person comprising the steps of:
selecting an appropriate target state;
filtering a play list of music tracks; and
vectoring by further filtering the play list to allocate the music tracks to play in order of appropriate stages creating a progressive modulation of the selected state from an initial state to the target state, wherein said music tracks are sorted by music tempo in beats per minute.

4. The method of claim 2, wherein the target state is selected from the group consisting of to help get them going, for motivation, to decrease fatigue, to relieve symptoms, improve sleep, and any other desired state the user wishes to change by using music.

5. A computer-implemented method of organizing music tracks based on music tempo in beats per minute comprising the steps of:
selecting a vector pattern;
filtering a play list of music tracks; and
vectoring by further filtering the play list to allocate the music tracks to play in order of appropriate stages creating a change in music tracks according to user preference, wherein said music tracks are sorted by music tempo in beats per minute.

6. A method of administering a computer-implemented methodology to modulate moods, symptoms, fatigue, and sleep quality of a patient undergoing cancer treatment comprising the steps of:
selecting an appropriate target mood;
filtering a play list of music tracks; and
vectoring by further filtering the play list to allocate the music tracks to play in order of appropriate stages creating a progressive modulation from an initial mood to the target mood, wherein said music tracks are sorted by music tempo in beats per minute.

7. The method of claim 6, further comprising the steps of:
monitoring and allow self-assessment of psychological responses in the patient.

8. The method of claim 7, wherein the monitoring of psychological responses further comprises measuring the patient's modulations in mood, symptoms, fatigue, and sleep disturbances.

9. A method of modulating stress in a patient with a debilitating condition and having an initial mood comprising the steps of
a) determining a target mood for the patient;
b) filtering a play list of music tracks stored on a computer;
c) vectoring the play list by further filtering the play list to allocate the music tracks for a progressive modulation of mood from the initial mood to the target mood;
d) monitoring psychological responses to determine stress level; and
e) repeating steps a through d until the target mood is reached.

10. The method of claim 9, wherein the debilitating condition is selected from the group consisting of: depression, cancer, Alzheimer's, Parkinson's, anxiety, dementia, stress, and tension.

11. A computer-readable medium containing instructions for controlling a computer system to carry out a method to modulate a mood of a person, the method comprising:
selecting a target mood;
filtering a play list of music tracks;
vectoring by further filtering the play list to allocate the music tracks to play in order of appropriate stages creating a progressive modulation of mood from an initial mood to the target mood, wherein said music tracks are sorted by music tempo in beats per minute.

12. A computer-readable medium containing instructions for controlling a computer system to carry out a method to organize music according to user preference for some desired target state, the method comprising:
selecting the target state;
filtering a play list of music tracks;
vectoring by further filtering the play list to allocate the music tracks to play in order of appropriate stages creating a progressive modulation of state from an initial state to the desired target state, wherein said music tracks are sorted by music tempo in beats per minute.

13. A computer-readable medium containing instructions for controlling a computer system to carry out a method to organizing music tracks based on music tempo in beats per minute, the method comprising:
selecting a vector pattern;
filtering a play list of music tracks; and
vectoring by further filtering the play list to allocate the music tracks to play in order of appropriate stages creating a change in music tracks according to user preference, wherein said music tracks are sorted by music tempo in beats per minute.

14. A computer-readable medium containing instructions for controlling a computer system to carry out a method to modulate a mood of a patient with a debilitating condition, the method comprising:
selecting a target mood;
filtering a play list of music tracks;
vectoring by further filtering the play list to allocate the music tracks to play in order of appropriate stages creating a progressive modulation from an initial mood to the target mood, wherein said music tracks are sorted by music tempo in beats per minute.

15. A program storage device readable by a computer, embodying a program of instructions executable by the computer to perform the steps for modulating a mood a person wherein said program comprises the steps of
selecting an appropriate target mood;
filtering a play list of music tracks;
vectoring by further filtering the play list to allocate the music tracks to play in order of appropriate stages creating a progressive modulation of mood from an initial mood to the target mood, wherein said music tracks are sorted by music tempo in beats per minute.

16. A program storage device readable by a computer, embodying a program of instructions executable by the computer to organize music according to user preference for some desired target state, wherein said program comprises the steps of:
selecting an appropriate target;
filtering a play list of music tracks;
vectoring by further filtering the play list to allocate the music tracks to play in order of appropriate stages creating a progressive modulation of a state from an initial state to the desired target state, wherein said music tracks are sorted by music tempo in beats per minute.

17. A program storage device readable by a computer, embodying a program of instructions executable by the computer to organize music tracks based on music tempo in beats per minute, wherein said program comprises the steps of:
selecting a vector pattern;
filtering a play list of music tracks;
vectoring by further filtering the play list to allocate the music tracks to play in order of appropriate stages creating a change in music tracks according to user preference, wherein said music tracks are sorted by music tempo in beats per minute.

18. A computer-implemented method of organizing music tracks with a user interface comprising:
providing for a display of program types, wherein the program types comprise:
an automatic program;
a semi-automatic program;
a manual play program;
an exercise program; and
a therapy program;
providing for selection of one of the program types by a user interface means; and
providing for instruction to execute the selected program type, wherein the music tracks are played in an organized manner according to user requirements and objectives.

19. The method of claim 18, wherein the automatic program comprises:
choosing a vector pattern from a plurality of pre-defined patterns based on BPM;
providing for retrieval of available music tracks to fit the vector pattern;
providing for automatic assignment of music tracks to said music pattern; and
providing for instruction to play the music tracks ensuring that the order of the music tracks follows the chosen vector pattern based on BPM of the music tracks.

20. The method of claim 18, wherein the semi-automatic program comprises:
choosing a vector pattern from a plurality of pre-defined patterns based on BPM;
providing for retrieval of available music tracks to fit the vector pattern;
providing for the user to choose music tracks to fit the vector pattern; and
providing for instruction to play the music tracks wherein the order of the music tracks follows the chosen vector pattern based on BPM of the music tracks.

21. The method of claim 18, wherein the manual play program comprises:
providing for retrieval of available music tracks based on BPM;
providing for addition of music tracks to a play list;
providing for inquiry of completion of the play list; and
providing for instruction to play the play list wherein the order of the music tracks follows the chosen order based on BPM of the music tracks.

22. The method of claim 18, wherein the exercise program comprises:
providing for retrieval of available music tracks;
providing for selection of a routine type, wherein the routine type is chosen from a custom routine and a plurality of pre-defined routines with different vector patterns based on BPM;
wherein the custom routine comprises:
providing for addition of music tracks to a play list based on BPM;
providing for inquiry of completion of the play list; and
providing instruction to play the play list wherein the order of the music tracks follows the chosen pattern based on BPM of the music tracks; and
wherein the plurality of pre-defined routines comprises filtering music tracks based on BPM to fit different vector patterns based on BPM:
providing for a warm up routine;
providing for a running routine; and
providing for a high impact routine; and
providing for execution of the selected routine wherein the order of the music tracks follows the chosen vector pattern based on BPM of the music tracks.

23. The method of claim 18, wherein the therapy program comprises the steps of:
providing for selection of an appropriate target mood;
providing for instruction to filter a play list of music tracks; and
providing for instruction to vector by further filtering the play list to allocate the music tracks to play in order of appropriate stages creating a progressive modulation of mood from an initial mood to the target mood, wherein the music tracks are sorted by music tempo in beats per minute.

24. The method of claim 23, where the program can be executed in one of three modes: 1) fully automatic where the music tracks are chosen by the program to fit a plurality of pre-defined vector patterns, 2) sem-automatic where the user will choose the music tracks to fit a plurality of pre-defined vector patterns, and 3) manual where the user will choose the vector pattern and the music tracks.

25. A computer-readable medium containing instructions for controlling a computer system with a user interface to carry out a method of organizing music tracks, the method comprising the steps of
providing for a display of program types, wherein the program types comprise:
an automatic program;
a semi-automatic program;
a manual play program;
an exercise program; and
a therapy program; and
providing for selection of one of the program types by a user interface means, wherein the music tracks are played in an organized manner according to user requirements and objectives.

26. The method of claim 18, wherein the automatic program comprises:
choosing a vector pattern from a plurality of pre-defined patterns based on BPM;
providing for retrieval of available music tracks to fit the vector pattern;
providing for automatic assignment of music tracks to said music pattern; and
providing for instruction to play the music tracks, wherein the order of the music tracks follows the chosen vector pattern based on BPM of the music tracks.

27. The method of claim 25, wherein the semi-automatic program comprises:
providing for retrieval of available music tracks based on a plurality of pre-defined patterns based on BPM;
choosing a vector pattern from a plurality of pre-defined patterns based on BPM;
providing for retrieval of available music tracks to fit the vector pattern;
providing for the user to choose music tracks to fit the vector pattern; and
providing for instruction to play the music tracks wherein the order of the music tracks follows the vector pattern based on BPM of the music tracks.

28. The method of claim 25, wherein the manual play program comprises:
providing for retrieval of available music tracks based on BPM;
providing for addition of music tracks to a play list;
providing for inquiry of completion of the play list; and
providing for instruction to play the play list wherein the order of the music tracks follows the chosen order based on BPM of the music tracks.

29. The method of claim 25, wherein the exercise program comprises:
providing for retrieval of available music tracks;
providing for selection of a routine type, wherein the routine type is chosen from a custom routine and a plurality of pre-defined routines with different vector patterns based on BPM;
wherein the custom routine comprises:
providing for addition of music tracks to a play list based on BPM;
providing for inquiry of completion of the play list; and
providing instruction to play the play list wherein the order of the music tracks follows the chosen order based on BPM of the music tracks; and
wherein the plurality of pre-defined routines comprises filtering music tracks based on BPM to fit different vector patterns based on BPM:
providing for a warm up routine;
providing for a running routine; and
providing for a high impact routine; and
providing for execution of the selected routine wherein the order of the music tracks follows the vector pattern based on BPM of the music tracks.

30. The method of claim 25, wherein the therapy program comprises the steps of:
providing for selection of an appropriate target mood;
providing for instruction to filter a play list of music tracks; and
providing for instruction to vector by further filtering the play list to allocate the music tracks to play in order of appropriate stages creating a progressive modulation of mood from an initial mood to the target mood, wherein the music tracks are sorted by music tempo in beats per minute.

31. The method of claim 30, where the program can be executed in one of three modes: 1) fully automatic where the music tracks are chosen by the program to fit a plurality of pre-defined vector patterns, 2) sem-automatic where the user will choose the music tracks to fit a plurality of pre-defined vector patterns, and 3) manual where the user will choose the vector pattern and the music tracks.

32. A program storage device readable by a computer equipped with a user interface, embodying a program of instructions executable by the computer to perform the steps for organizing music tracks wherein the program comprises the steps of:
providing for a display of program types, wherein the program types comprise:
an automatic program; a semi-automatic program;
a manual play program;
an exercise program; and
a therapy program; and
providing for selection of one of the program types by a user interface means, wherein the music tracks are played in an organized manner according to user requirements and objectives.

33. The method of claim 32, wherein the automatic program comprises:
choosing a vector pattern from a plurality of pre-defined patterns based on BPM;
providing for retrieval of available music tracks to fit the vector pattern;
providing for automatic assignment of music tracks to said music pattern; and
providing for instruction to play the music tracks wherein the order of the music tracks follows the vector pattern based on BPM of the music tracks.

34. The method of claim 32, wherein the semi-automatic program comprises:
choosing a vector pattern from a plurality of pre-defined patterns based on BPM;
providing for retrieval of available music tracks to fit the vector pattern;
providing for the user to choose music tracks to fit the vector pattern; and
providing for instruction to play the music tracks wherein the order of the music tracks follows the vector pattern based on BPM of the music tracks.

35. The method of claim 32, wherein the manual play program comprises:
providing for retrieval of available music tracks based on BPM;
providing for addition of music tracks to a play list;
providing for inquiry of completion of the play list; and
providing for instruction to play the play list wherein the order of the music tracks follows the chosen order based on BPM of the music tracks.

36. The method of claim 32, wherein the exercise program comprises:
providing for retrieval of available music tracks;
providing for selection of a routine type, wherein the routine type is chosen from a custom routine and a plurality of pre-defined routines with different vector patterns based on BPM:
wherein the custom routine comprises:
providing for addition of music tracks to a play list based on BPM;
providing for inquiry of completion of the play list; and
providing instruction to play the play list wherein the order of the music tracks follows the chosen order based on BPM of the music tracks; and
wherein the plurality of pre-defined routines comprises filtering music tracks based on BPM to fit different vector patterns based on BPM:
providing for a warm up routine;
providing for a running routine; and
providing for a high impact routine; and
providing for execution of the selected routine wherein the order of the music tracks follows the vector pattern based on BPM of the music tracks.

37. The method of claim 32, wherein the therapy program comprises the steps of:
providing for selection of an appropriate target mood;
providing for instruction to filter a play list of music tracks; and
providing for instruction to vector by further filtering the play list to allocate the music tracks to play in order of appropriate stages creating a progressive modulation of mood from an initial mood to the target mood, wherein the music tracks are sorted by music tempo in beats per minute.

38. The method of claim 37, where the program can be executed in one of three modes: 1) fully automatic where the music tracks are chosen by the program to fit a plurality of pre-defined vector patterns, 2) sem-automatic where the user will choose the music tracks to fit a plurality of pre-defined vector patterns, and 3) manual where the user will choose the vector pattern and the music tracks.

39. A method of treating a patient with a debilitating condition by administering to the patient a computer-implement methodology comprising the steps of
a) determining an initial mood of the patient;
b) determining a target mood for the patient;
c) filtering a play list of music tracks stored on a computer;
d) vectoring the play list by further filtering the play list to allocate the music tracks for a progressive modulation of mood from the initial mood to the target mood;
e) monitoring psychological responses to determine stress level; and
f) repeating steps b through e until the target mood is reached.

40. The method of claim 39, wherein the debilitating condition is selected from the group consisting of: depression, cancer, Alzheimer's, Parkinson's, anxiety, dementia, stress, and tension.
